(19) United States
(12) Patent Application Publication
(10) Pub. No.: US 2025/0231137 A1
(43) Pub. Date: Jul. 17, 2025

(54) COMPOUND DELIVERY PROCESSES & DEVICES TO MEASURE ELECTRICAL SIGNALS OF MATERIAL DISPOSED IN FLUID

(71) Applicant: Analog Devices International Unlimited Company, Limerick (IE)

(72) Inventors: David Duncan Lloyd, Menlo Park, CA (US); Phillip Michel Nadeau, Cambridge, MA (US); Junfei Xia, Andover, MA (US); Michael C.W. Coln, Lexington, MA (US); Roman Trogan, Needham, MA (US)

(21) Appl. No.: 19/030,260

(22) Filed: Jan. 17, 2025

Related U.S. Application Data
(60) Provisional application No. 63/621,956, filed on Jan. 17, 2024.

(51) Int. Cl. G01N 27/327; G01N 33/543

(52) U.S. Cl. CPC G01N 27/3276; G01N 33/5438

(57) ABSTRACT

Semiconductor devices can include features to apply electrical stimulation to liquids in which one or more compounds are present and to measure the response to the electrical stimulation. Nucleic acids can be used to track the location of the compounds within the semiconductor devices.

### PRIORITY CLAIM

This application claims priority to U.S. provisional patent application No. 63/621,956 filed Jan. 17, 2024, and entitled Compound Delivery Methods for an Automated Patch Clamp System, which is incorporated by reference herein in its entirety.

### FIELD OF THE DISCLOSURE

This document pertains generally, but not by way of limitation, to apparatuses and methods related to delivering fluids to sites of semiconductor devices sites to measure the electrical response of material located in the sites.

### BACKGROUND

Electrical stimulation can involve applying at least one of current or voltage to a substance. The response of the substance to the electrical stimulation can indicate characteristics of the substance. For example, an amount of conductivity of a substance can be determined in response to applying one or more electrical stimuli to the substance. In various examples, changes in electrical properties of a substance in response to one or more electrical stimuli can also indicate characteristics of the substance. To illustrate, some substances can undergo changes in composition and/or changes in form in response to one or more electrical stimuli. In one or more scenarios, the response of substances to electrical stimulation can be measured when the substances are disposed in fluids. In some examples, an automated patch clamp system can cause electrical stimuli to be applied to biological cells disposed in fluid. The electrical response of the biological cells to the electrical stimuli can indicate characteristics of the biological cells.

### SUMMARY OF THE DISCLOSURE

In one or more examples, a method comprises providing a semiconductor device including a substrate having one or more layers and an array of sites formed on the substrate. Individual sites of the array of sites can include a number of walls and the one or more layers of the substrate can include a complementary metal oxide semiconductor (CMOS) layer. The method can also include forming one or more first nucleic acid molecules within individual sites of the array of sites. Individual nucleotide sequences of the one or more first nucleic acid molecules can include an identifier of a location of an individual site within the array of sites. In addition, the method can include dispensing a solution on the semiconductor device. The solution can include a plurality of substrates with one or more second nucleic acid molecules coupled to an individual substrate and one or more test compounds being coupled to the individual substrate. The one or more second nucleic acid molecules can include nucleotide sequences that include an identifier of the one or more test compounds. Further, the method can include providing a number of biological cells to the semiconductor device and applying at least one of voltage or current to one or more electrodes included in the individual sites of the array of sites while the one or more test compounds and one or more biological cells of the number of biological cells are disposed in the individual sites. Additionally, the method can include measuring electrical signals produced in response to the at least one of voltage or current being applied to the plurality of electrodes.

In one or more examples, an article can comprise a bead and one or more instances of a compound can be bound to the bead. The compound can have an average molecular weight that is no greater than 1000 grams/mol. In addition, one or more nucleic acids can be bound to the bead. The one or more nucleic acids can include a segment having a nucleotide sequence that corresponds to an identifier of the compound.

In one or more examples, a device can comprise an outer structure including a first segment, a second segment that is joined to the first segment and disposed at least substantially perpendicular with respect to the first segment, and a third segment that is joined to the third segment and is disposed at least substantially parallel with respect to the first segment. The device can also comprise an inner structure including a first additional segment that is disposed at least substantially parallel with respect to the first segment and a second additional segment that is disposed at least substantially perpendicular with respect to the first additional segment. The first segment and the first additional segment can form a first portion of a channel and the second segment and the second additional segment can form a second portion of the channel that is disposed at least substantially perpendicular with respect to the first portion of the channel. In addition, an opening can be disposed in the second segment and can be in fluid communication with the second portion of the channel. Further, an excess fluid area can be disposed above the channel and can be formed by the first additional segment and the second additional segment of the inner structure and the third segment of the outer structure.

In one or more examples, a method comprises providing a fluid dispensing device including a plurality of sections with individual sections of the plurality of sections including an outer structure and an inner structure. The outer structure and the inner structure form a channel and an inlet for the channel. The inlet is disposed at least substantially perpendicular with respect to the channel and the channel includes an opening to dispense liquid. The method can also include loading a discrete amount of a plurality of liquids within the channel of the individual sections via the inlet of the individual sections and providing a fluid accepting device including a number of sites. Individual sites of the number of sites can be configured to store an amount of at least one liquid of the plurality of liquids. Additionally, the method can include causing a first group of sites of the number of sites to be aligned with individual openings of the individual sections and causing one or more first liquids of the plurality of liquids to be dispensed from the individual openings into the first group of sites. Further, the method can include causing a second group of sites of the number of sites to be aligned with the individual openings of the individual sections and causing one or more second liquids of the plurality of liquids to be dispensed from the individual openings into the second group of sites.

In one or more examples, a semiconductor device comprises a substrate having one or more layers and an array of sites formed on the substrate. The one or more layers of the substrate can include a complementary metal oxide semiconductor (CMOS) layer. Individual sites can include a number of walls comprised of one or more hydrophobic materials. The individual sites can also include a base surface comprised of one or more hydrophilic materials. The base surface can include a first portion having one or more first materials to couple one or more nucleic acids to the first section and a second portion having one or more second materials to couple a bead to the second section.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various implementations discussed in the present document.

FIG. 1 is a diagram depicting a framework for applying electrical stimulation to substances disposed in liquid using semiconductor devices, in accordance with one or more example implementations.

FIG. 2 is a diagram depicting a cross-section of at least a portion of an example semiconductor device in which at least one of current or voltage can be applied to substances disposed in liquid, in accordance with one or more example implementations.

FIG. 3 is a diagram depicting a top view of a pattern of sites of a semiconductor device in which electrical signals are applied to substances disposed in the sites, in accordance with one or more example implementations.

FIG. 4 is a diagram depicting a process to prepare semiconductor device including an array of sites to test the electrical response of compounds disposed in individual sites, in accordance with one or more example implementations.

FIG. 5 is a diagram depicting a process to track the electrical response of compounds disposed in sites of a semiconductor device, in accordance with one or more implementations.

FIG. 6 is a diagram depicting a process to use nucleic acid molecules to track the electrical response of compounds disposed in sites of a semiconductor device, in accordance with one or more example implementations.

FIG. 7 is a diagram depicting a cross-sectional view of a first example fluid dispensing device to deposit fluids within an array of sites included in a semiconductor device, in accordance with one or more example implementations.

FIG. 8A is a diagram depicting a top view of an example fluid dispensing device, in accordance with one or more example implementations.

FIG. 8B is a view of the example fluid dispensing device and a fluid accepting device.

FIG. 9 is a diagram depicting a top view and a cross-sectional view of a second example fluid dispensing device to deposit fluids within an array of sites included in a semiconductor device, in accordance with one or more example implementations.

FIG. 10 is a diagram depicting a cross-sectional view of an additional example outlet arrangement for a device that dispenses fluid to an array of sites in a semiconductor device, in accordance with one or more example implementations.

FIG. 11 is a flow diagram of a process to dispense fluids from a fluid accepting device to a fluid accepting device, in accordance with one or more example implementations.

### DETAILED DESCRIPTION

Electrophysiology can include applying electrical stimulation to biological material and measuring changes to electrical properties of the biological material in response to the electrical stimulation to identify one or more characteristics of the biological material. In some cases, electrophysiology can be implemented to determine one or more measures of functionality of the biological material. (e.g., health of tissue or cells) In other scenarios, electrophysiology can be implemented to determine measures of functionality of the biological material in a given environment. For example, the responsiveness of cells to one or more compounds or therapeutics can be measured.

In one or more illustrative examples, patch clamp electrophysiology is a technique that can be used in various fields of medicine and biology for interrogating electrogenic cells such as neurons and cardiomyocytes. Several diseases originate from the electrical properties of these cells, especially diseases of ion channels, or so-called channelopathies comprising neurological disorders, cardiac disorders, pain disorders, and more. Patch clamp enables the study of electrogenic cells by allowing direct control and measurement of cellular membrane potential and ion channel currents. Within fundamental science, patch clamp can be used to understand the basic mechanisms of cell function and disease. Within the study of disease, it can be used to probe specific disease mechanisms and discover molecules that deliver effective treatments.

Manual patch clamp is an approach to perform patch clamp electrophysiology using humancentric equipment and workflows. An example of such an experiment can involve manually bringing a micron-scale glass pipette to the surface of a cell under microscope observation, optionally performing a brief suction through the pipette to disrupt the cell membrane and gain access inside the cell, and then performing current clamp, voltage clamp, dynamic clamp, or other electrical measurement to probe the electrical workings of the cell. Despite skill arguably being the gold standard measurement in many contexts, the technique is very labor intensive and time consuming, involving hours-to-days to perform each measurement.

Automated patch clamp (APC) techniques can help improve the throughput and reduce the cost of performing patch clamp measurements. One class of approaches can be described as direct automation of the manual approach, deploying imaging technology to locate cells within a plate, and robotic technology to automatically manipulate the glass pipette to achieve the seal and measurements. Such techniques can reduce the reliance on a human operator, but do not scale the throughput dramatically.

Another class of approaches can be built around large robotic workstations with liquid handling functions. The robotic head can be used to pipette cells into individual chambers, which can be wells in a well-plate, or customized volumes with additional flow features. Those chambers can feature micron-scale pores that can trap cells under suction and can also open access into cells with additional suction. The electrical measurements can then be performed using a set of discrete electronics, such as multiplexed to electrodes near the chambers. These techniques can dramatically increase the throughput up to 384 independent readouts and can push beyond this.

There is a need for even higher throughput and lower cost. The area of drug discovery for Central-Nervous-System (CNS) disorders has seen extraordinarily low productivity in recent years, leaving many diseases without effective treatments. Most commercial small-molecule screening libraries number in the millions of compounds, and full-file screens with a hundred-site-scale automated patch clamp instrumentation are prohibitive. An increased capacity to screen libraries against electrogenic cells can be a tool to power discoveries, such as in the CNS area.

In order to match the results of electrical tests with corresponding compounds, the location of the compounds within devices used to perform the electrical tests is determined. In situations where the location of the compounds within the devices is not known, matching electrical test results with individual compounds may not be possible. Determining compounds at locations of a testing device can be done by the deterministic transfer of compounds, wherein the compounds are deposited in known locations. Alternatively, a non-deterministic transfer process can be used, in which the compounds are deposited to non-predetermined locations, with their locations subsequently identified. The advantage of non-deterministic transfer methods is that it lessens the need for precise control of where individual compounds are placed.

In some scenarios, the same compound can be delivered to each site of a testing device. In additional instances, the location of compounds within a testing device can be tracked by distributing individual amounts of fluid to individual sites of the testing device and monitoring the compounds included in the individual droplets. In various examples, the location of compounds within sites of a testing device can be tracked by loading a fluid dispensing device with a compound from a compound source and then dispensing droplets including the compound in a portion of the sites of the testing device. The sites into which the droplets are dispensed can be tracked based on determining the alignment of the outlets of the fluid dispensing device with the locations of the sites. Additional compounds can be delivered to additional sites of the testing device by loading the same fluid dispensing device or a different fluid dispensing device with a different compound from a different source. In various implementations described herein, fluid dispensing devices are designed such that multiple compounds can be delivered to sites of a testing device by loading multiple compounds into the fluid dispensing device and then dispensing the compounds into sites of an array of sites according to a given arrangement or protocol. In this way, the efficiency of the process of delivering compounds to a testing device can be increased and the time and resources used to deliver compounds to testing devices can be decreased.

In various additional implementations, multiple compounds can be delivered to a testing device in a bulk process. For example, compounds to be tested can be added to a fluid that is then distributed in bulk to the testing device. To illustrate, the fluid including the compounds can be distributed in a stream onto the testing device. In these scenarios, the precise location of different compounds in sites of the testing device may not be controlled during the fluid dispensing process. In one or more examples, the locations of the compounds within the individual sites of the testing device can be determined by using molecules that identify both the site of the testing device where the individual compounds are located and that identify the individual compounds disposed in the individual sites.

In one or more examples, nucleic acid molecules can be used to identify both the individual sites of a testing device and the compounds included in the individual sites. For example, individual first nucleotide sequences can be determined that correspond to individual sites of the testing device. Additionally, second nucleotide sequences can be determined that correspond to individual compounds being tested using the testing device. In various examples, first nucleic acid molecules can be synthesized having the first nucleotide sequences. In one or more illustrative examples, the first nucleic acid molecules can be synthesized within the individual sites and attached to a surface of the individual sites by one or more first linker molecules. In one or more additional examples, second nucleic acid molecules can be synthesized having the second nucleotide sequences. The second nucleic acid molecules can be bound to individual substrate in conjunction with the individual compounds being tested. The substrates with the second nucleic acid molecules and individual compounds bound to the substrates can be delivered to the sites of the testing device. In at least some examples, the substrates with the bound compounds and second nucleic acid molecules can be dispensed in a stream along a surface of the testing device to deposit the substrates into the individual sites. In one or more further examples, biological cells can also be deposited into the sites of the testing device.

After depositing the substrates in conjunction with the second nucleic acid molecules and the compounds, the compounds and the second nucleic acid molecules can be released from the substrate. Electrical signals can be applied to the sites of the testing device and the response to the electrical signals can be measured. The second nucleic acid molecules can then be bound with the first nucleic acid molecules and double stranded nucleic acid molecules can be synthesized from the hybridized first nucleic acid molecules and the second nucleic acid molecules. By combining the first nucleic acid molecules and the second nucleic acid molecules, the double stranded nucleic acid molecules can include an identifier of the site in which the first nucleic acid molecule was deposited and an identifier of the compound located in the site. Sequencing operations can be performed to determine the nucleotide sequences of one or more strands of the double stranded nucleic acid molecules to determine the compounds paired with the individual sites of the testing device. The electrical response measured for individual sites of the testing device can then be identified and the impact of the compound on cells deposited in the sites can be determined based on the electrical response.

In one or more implementations, high-throughput patch clamp systems can be manufactured using technologies different from existing techniques. For example, high-throughput patch clamp systems can be manufactured using semiconductor technologies. In this way, the components used to perform patch clamp measurements can be produced on a scale that is smaller than that of existing patch clamp systems. Accordingly, techniques described herein can be used to produce patch clamp systems having a greater number of sites for performing patch clamp measurements than existing techniques and results in higher throughput than existing techniques. In this way, the speed of the development of therapeutic compounds can be increased because more compounds can be tested at a single time than with existing techniques. Because more candidate therapeutic compounds can be tested at a single time, the possibility of identifying viable therapeutic compounds to treat a given biological condition also increases.

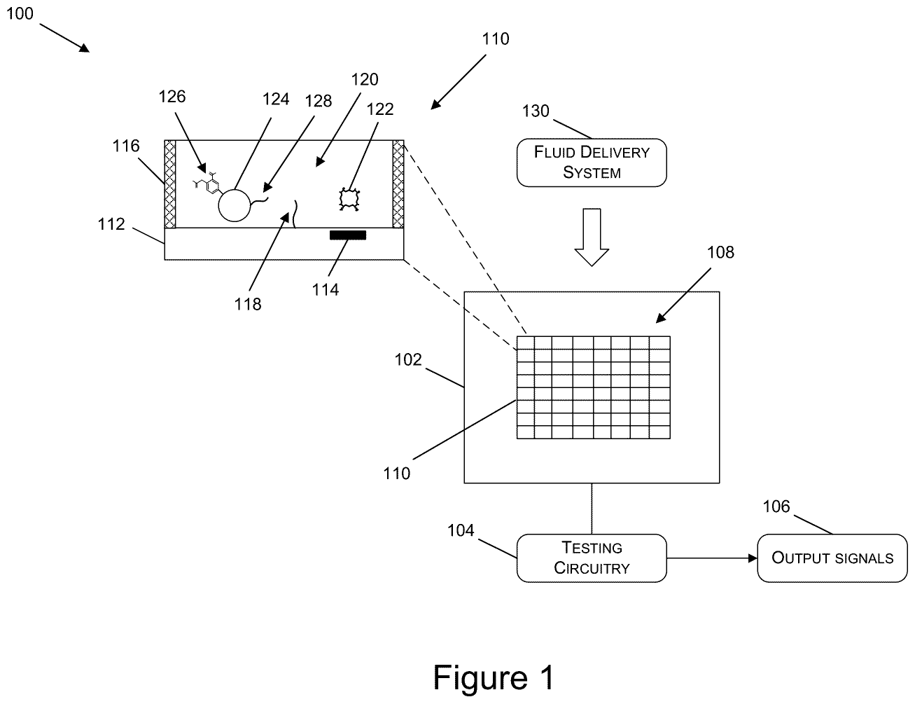

FIG. 1 is a diagram depicting a framework 100 for applying electrical stimulation to substances disposed in liquid using semiconductor devices, in accordance with one or more example implementations. The framework 100 can include a semiconductor device 102. The semiconductor device 102 can be comprised of one or more silicon-containing materials. For example, the semiconductor device 102 can be comprised of at least one of silicon, silicon nitride, or polycrystalline silicon. The semiconductor device 102 can include a die, also referred to herein as a chip, that includes circuitry to apply at least one of voltage or current to liquid disposed in or disposed on the die. The die can also include circuitry to measure signals produced in response to applying at least one voltage or current to the liquid. In various examples, biological material can be disposed in the liquid. In these scenarios, the signals produced in response to applying voltage and/or current to the liquid can indicate one or more characteristics of the biological material.

In one or more examples, the die can include a silicon-containing substrate with circuitry formed on an outer surface of the die. In at least some additional examples, the die can include a silicon-containing substrate with a plurality of layers and circuitry formed on and/or formed within at least a subset of the plurality of layers. The circuitry can be formed according to one or more complementary metal-oxide semiconductor (CMOS) technologies. In one or more illustrative examples, the semiconductor device 102 can include or be coupled to testing circuitry 104. The testing circuitry 104 can cause at least one of voltage or current to be applied to fluid disposed on the semiconductor device 102 and generate output signals 106 in response to the electrical stimulation. In one or more additional illustrative examples, the testing circuitry 104 can perform at least one of a voltage clamp process, a current clamp process, or a dynamic clamp process.

The output signals 106 can correspond to an electrical response of biological material located in the fluid to the voltage and/or current stimuli. In at least some examples, the semiconductor device 102 can store at least a portion of the output signals 106. In one or more additional examples, the semiconductor device 102 can include additional circuitry to analyze the output signals 106. In one or more further examples, the semiconductor device 102 can include hardware processing resources and memory with the hardware processing resources being configured to execute computer-readable instructions to analyze the output signals 106.

In various examples, the testing circuitry 104 can implement one or more optical stimulation protocols. For example, the testing circuitry 104 can cause electromagnetic radiation to be applied to fluid disposed on the semiconductor device 102. In one or more additional examples, an electromagnetic radiation source can be external to the semiconductor device 102. In one or more illustrative examples, the testing circuitry 104 can include one or more optical filters to provide electromagnetic radiation having one or more specified wavelengths to fluid disposed on the semiconductor device 102. The testing circuitry 104 can measure an optical response of fluid and/or substances disposed in fluid located on the semiconductor device 102. In various examples, the testing circuitry 104 can produce output signals 106 that correspond to a fluorescence response to optical stimulation by at least one of fluids or substances disposed in fluids located on the semiconductor device 102.

The die can be disposed in packaging of the semiconductor device 102. The packaging can be comprised of one or more polymeric materials. Additionally, the packaging can be comprised of one or more metallic materials. Further, the packaging can be comprised of one or more ceramic materials. In one or more examples, the packaging can include circuitry coupled to the circuitry of the die. In at least some examples, the semiconductor device 102 can include one or more routing layers to carry electrical signals from circuitry formed on the die to circuitry formed on the packaging.

The semiconductor device 102 can also include circuitry to directly or indirectly couple the die to one or more external computing devices. In these instances, signals generated by circuitry on the die can be sent to the one or more external computing devices. in one or more illustrative examples, the semiconductor device 102 can include circuitry to wirelessly communicate information to one or more external computing devices, such as via a Bluetooth communication protocol or an Institute of Electrical and Electronics Engineers (IEEE) 802.11 communications protocol. In at least some scenarios, the one or more external computing devices can analyze the signals produced by the circuitry on the die to determine one or more characteristics of at least one of fluid or biological materials disposed on the semiconductor device 102.

The semiconductor device 102 can include an array of sites 108. The array of sites 108 can be formed on and/or can be formed by the die of the semiconductor device 102. Individual sites of the array of sites 108 can include components to apply at least one of voltage or current to fluid disposed in the individual site and to measure signals produced in response to the voltage and/or current applied to the fluid. In one or more examples, the sites of the array of sites 108 can be arranged in rows and columns. In various examples, the rows of sites can be in fluid communication with one another via one or more channels formed in the die of the semiconductor device 102. In one or more additional examples, the columns of sites can be in fluid communication with one another via one or more channels formed in the die of the semiconductor device 102. In one or more illustrative examples at least 10 channels, at least 50 channels, at least 100 channels, at least 250 channels, at least 500 channels, at least 1000 channels, at least 2000 channels, at least 3000 channels, at least 4000 channels, at least 5000 channels, at least 6000 channels, at least 7000 channels, at least 8000 channels, at least 9000 channels, at least 10,000 channels, or more can be formed in the die of the semiconductor device 102 to couple sites of the array of sites 108. In one or more additional illustrative examples, from 10 channels to 25,000 channels, from 100 channels to 20,000 channels, from 1000 channels to 15,000 channels, from 5000 channels to 10,000 channels, from 1000 channels to 10,000 channels, from 100 channels to 1000 channels, from 10 channels to 100 channels, from 2000 channels to 8000 channels, or from 8000 channels to 12,000 channels can be formed in the dies of the semiconductor device 102 to couple sites of the array of sites 108.

In various examples, the array of sites 108 can include at least 5 sites, at least 20 sites, at least 50 sites, at least 100 sites, at least 200 sites, at least 500 sites, at least 1000 sites, at least 2000 sites, at least 3000 sites, at least 4000 sites, at least 5000 sites, at least 6000 sites, at least 7000 sites, at least 8000 sites, at least 9000 sites, at least 10,000 sites, at least 11,000 sites, at least 12,000 sites, at least 13,000 sites, at least 14,000 sites, at least 15,000 sites, or more. To illustrate, the array of sites 108 can include from 5 sites to 100,000 sites, from 10 sites to 50,000 sites, from 100 sites to 20,000 sites, from 1000 sites to 10,000 sites, from 10,000 sites to 50,000 sites, from 10,000 sites to 30,000 sites, from 5000 sites to 10,000 sites, from 8000 sites to 15,000 sites, from 1000 sites to 5000 sites, from 100 sites to 1000 sites, from 5 sites to 100 sites, from 500 sites to 2000 sites, or from 2000 sites to 8000 sites.

The array of sites 108 can have a rectangular shape, a circular shape, or an ellipsoidal shape. In one or more examples, the array of sites 108 can have a surface area from about 0.1 cm² to about 25 cm², from about 0.5 cm² to about 15 cm², from about 1 cm² to about 10 cm², from about 2 cm² to about 8 cm², from about 3 cm² to about 10 cm², from about 1 cm² to about 5 cm², from about 0.1 cm² to about 1 cm², from about 4 cm² to about 10 cm², or from about 8 cm² to about 15 cm². In illustrative scenarios where the array of sites 108 has a rectangular shape, the array of sites 10l can have a length and a width. In these instances, the array of sites 108 can have at least one of a length or width from about 0.1 cm to about 5 cm, from about 0.5 cm to about 4 cm, from about 1 cm to about 3 cm, from about 2 cm to about 5 cm, or from about 0.5 cm to about 2 cm.

Individual sites 110 of the array of sites 108 can include a number of features. For example, individual sites 110 of the array of sites 108 can include a support structure 112. The support structure 112 can include a number of substrates. In one or more examples, the support structure 112 can include a number of substrates of a die of the semiconductor device 102. In at least some examples, the support structure 112 can include one or more silicon-containing substrates. For example, the support structure 112 can include one or more substrates comprised at least primarily of silicon. In one or more additional examples, the support structure 112 can include one or more substrates comprised of at least one of silicon carbide or silicon nitride. In one or more further examples, the support structure 112 can include one or more substrates comprised of one or more polymeric materials. To illustrate, the support structure 112 can include one or more substrates comprised of one or more polyimides. In various examples, the support structure 112 can include one or more polycrystalline silicon layers. In still other examples, the support structure 112 can include one or more glass substrates. The support structure 112 can also comprise one or more passivation layers. The one or more passivation layers can be comprised of at least one of one or more oxide materials or one or more nitride materials. In one or more examples, the support structure 112 can include a layer comprised of one or more hydrophilic materials. Examples of hydrophilic materials included in one or more layers of the support structure 112 can include at least one of silicon oxide, silicon nitride, titanium oxide, or aluminum oxide.

Additionally, the support structure 112 can include one or more functional layers. The one or more functional layers can include circuitry to apply at least one of voltage or current to fluid disposed in the individual site 110 and to generate signals in response to the voltage and/or current being applied to the fluid. The support structure 112 can also include one or more bond pads and a number of thin film transistor (TFT) switches to control the flow of current to electrodes formed in the individual sites 110. In the illustrative example of FIG. 1, the individual site 110 includes an electrode 114. In various examples, the individual sites 110 can also include or associated with one or more reference electrodes.

In one or more examples, the site 110 can be formed by a plurality of walls 116. The plurality of walls 116 can be comprised of one or more polymeric materials. The plurality of walls 116 can also be comprised of one or more silicon-containing materials. In one or more illustrative examples, the plurality of walls 116 can be comprised of a photoresist material. For example, the plurality of walls 116 can be comprised of an epoxy-based photoresist. To illustrate, the plurality of walls 116 can be comprised of an SU-8 photoresist. In one or more additional illustrative examples, the plurality of walls 116 can be comprised of a polyimide-containing material. In one or more further examples, the plurality of walls 116 can be comprised of a polysilicon-containing material.

The site 110 can also include a first nucleic acid 118. The first nucleic acid 118 can be coupled to a surface of the support structure 112. In one or more examples, the first nucleic acid 118 can be coupled to the surface of the support structure 112 by one or more types of chemical bonding. For example, the first nucleic acid 118 can be coupled to the surface of the support structure 112 by at least one of covalent bonding, ionic bonding, hydrogen bonding, or van der Waals forces. In one or more additional examples, the first nucleic acid 118 can be coupled to the surface of the support structure 112 by a linker molecule that is bound to the surface of the support structure 112. Although the illustrative example of FIG. 1 shows a single nucleic acid coupled to the surface of the support structure 112, in other implementations, multiple nucleic acids can be bound to the surface of the support structure 112.

In various examples, a liquid 120 can be disposed in the site 110. In at least some examples, the liquid 120 disposed in the site 110 can be in the form of a droplet. One or more biological cells 122 can be disposed in the liquid 120. The one or more biological cells 122 can include biological material that is undergoing testing and/or experiments using the semiconductor device 102. In one or more illustrative examples, the one or more biological cells 122 can comprise electrogenic cells. In one or more additional illustrative examples, the one or more biological cells 122 can comprise optogenetic cells. In one or more further illustrative examples, the one or more biological cells 122 can include at least one of neurons or cardiomyocytes. In one or more illustrative examples, the sites 110 can be designed to electrically and fluidically isolate the one or more biological cells located in a first site of the array of sites 108 from additional sites included in the array of sites 108 during at least a portion of the process to apply electrical stimulation to the liquid 120 and measure the response to the electrical stimulation. For example, the structure of the sites 110 can cause the biological cells 122 to be at least one of electrically or fluidically isolated from biological cells included in additional sites. Further, materials forming surfaces of the sites 110 can be selected to electrically and fluidically isolate biological cells in one site of the array of sites 108 from other sites included in the array of sites 108. To illustrate, materials of the walls 116 and materials of the support structure 112 that form a floor of the sites 110, can be comprised of materials that result in the biological cells of one site to be at least one of fluidically or electrically isolated from biological cells of additional sites of the array of sites 108.

Additionally, the liquid 120 can also include a compound carrier 124. A number of molecules can be bound to the compound carrier 124. To illustrate, a compound 126 can be bound to the compound carrier 124. In one or more examples, the compound 126 can include a molecule that may impact the biological cell 122. In various examples, the compound 126 can be disposed within one or more sites of the array of sites 108 to determine whether or not the compound 126 causes modification to at least one of the function or structure of the biological cells 122 by measuring responses to electrical signals applied to the array of sites 108 when the biological cells 122 are in the presence of one or more compounds, such as the compound 126.

Further, second nucleic acid 128 can be bound to the compound carrier 124. The compound 126 and the second nucleic acid 128 can be bound to the compound carrier 124 by one or more types of chemical bonding. For example, at least one of the compound 126 or the second nucleic acid 128 can be coupled to the compound carrier 124 by at least one of covalent bonding, ionic bonding, hydrogen bonding, or van der Waals forces. In one or more examples, at least one of the compound 126 or the second nucleic acid 128 can be coupled to the compound carrier 124 by a linker molecule that is bound to the surface of the compound carrier 124. Although the illustrative example of FIG. 1 shows a single nucleic acid coupled to the compound carrier 124 and a single compound molecule coupled to the compound carrier 124, in other implementations, multiple nucleic acids and/or multiple compound molecules can be bound to the compound carrier 124.

In one or more examples, the compound carrier 124 can include a bead. In various examples, the compound carrier 124 can include a bead comprised of silica. In one or more additional examples, the compound carrier 124 can include a bead comprised of one or more polymeric materials. In one or more further examples, the compound carrier 124 can include a bead comprised of one or more metallic materials. In one or more illustrative examples, the compound carrier 124 can include a bead comprised of one or more metals having magnetic properties. In at least some examples, the compound carrier 124 can include a bead comprised of multiple materials. For example, the compound carrier 124 can include a bead having a metallic core with one or more polymeric layers encasing the metallic core.

The first nucleic acid 118 can include a first nucleotide sequence that corresponds to an identifier of the site 110. For example, the first nucleic acid 118 can have a first nucleotide sequence that indicates a location of the site 110 within the array of sites 108. Additionally, the second nucleic acid 128 can include a second nucleotide sequences that corresponds to an identifier of the compound 126. In at least some examples, the first nucleic acid 118 can have a first nucleotide sequence that uniquely identifies a location of the site 110 within the array of sites with respect to additional nucleotide sequences of additional nucleic acids that indicate locations of other sites within the array of sites 108. Further, the second nucleic acid 128 can have a second nucleotide sequence that uniquely identifies the compound 126 with respect to additional compounds that can be disposed in additional sites of the array of sites 108. In this way, the first nucleic acid 118 and the second nucleic acid 128 can serve as identifiers of the combination of the location of the site 110 within the array of sites and the compound 126 that is disposed in the site 110.

The liquid 120 can be disposed in the site 110 by a fluid delivery system 130. The fluid delivery system 130 can deliver a volume of liquid to the semiconductor device 102. In one or more examples, the fluid delivery system 130 can deliver discrete amounts of liquid to individual sites 110 of the array of sites 108. In one or more additional examples, the fluid delivery system 130 can supply a bulk amount of liquid to the array of sites 108. For example, the fluid delivery system 130 can at least one of spray or stream a volume of liquid onto the array of sites 108. In one or more further examples, the fluid delivery system 130 can supply a volume of liquid to the semiconductor device 102 that is distributed to individual sites 108 using a fluid distribution arrangement of the semiconductor device 102. To illustrate, the semiconductor device 102 can include fluid distribution channels to deliver an amount of liquid to the sites 110. In various examples, the fluid delivery system 130 can deliver liquid to the semiconductor device 102 in one or more batches. In at least some examples, the fluid delivery system 130 can provide a discrete volume of liquid to the semiconductor device 102 at a given time. In still other examples, the fluid delivery system 130 can continuously provide liquid to the semiconductor device 102 over a period of time.

In one or more examples, the fluid delivery system 130 can include a number of pipettes that are included in an automated liquid handling system to deliver liquid to at least a portion of the array of sites 108 of the semiconductor device 102. For example, the fluid delivery system 130 can include a robotic liquid handling system to provide liquid to the array of sites 108. In various examples, the semiconductor device 102 can include one or more pipette landing sites to receive liquid from the fluid delivery system 130. Additionally, the fluid delivery system 130 can include a number of pins on which droplets can be disposed. The droplets disposed on the pins can be released and supplied to the array of sites 108. In still other examples, the fluid delivery system 130 can include a one or more structures with one or more openings. Pressure can be applied to fluid stored in the one or more structures to cause at least a portion of the fluid to be dispensed to at least a portion of the array of sites 108.

During a cycle of applying at least one of voltage or current to the liquid 120 and measuring an electrical response of the liquid 120, the compound 126 can be released from the compound carrier 124. For example, a bond between the compound carrier 124 and the compound 126 can be cleaved. To illustrate, temperature changes, pH changes, electromagnetic radiation, one or more enzymes, or one or more combinations thereof, can be applied to the compound carrier 124 and the compound 126 to cause the compound 126 to be released from the compound carrier 124. In this way, the biological cell 122 can be in the presence of the compound 126 without the compound 126 being bound to the compound carrier 124. In at least some examples, the compound 126 can be released from the compound carrier 124 to enable interaction between the biological cell 122 and the compound 126.

Additionally, the second nucleic acid 128 can be released from the compound carrier 124 during a cycle of applying at least one of voltage or current to the liquid 120 and measuring an electrical response of the liquid 120. In one or more examples, a bond between the compound carrier 124 and the second nucleic acid 128 can be cleaved. To illustrate, temperature changes, pH changes, electromagnetic radiation, one or more enzymes, or one or more combinations thereof, can be applied to the compound carrier 124 and the second nucleic acid 128 to cause the second nucleic acid 128 to be released from the compound carrier 124. In various examples, one or more first modalities applied to the compound 126 and the compound carrier 124 to cause the compound 126 to be released from the compound carrier 124 can be different from one or more second modalities applied to the compound 126 and the second nucleic acid 128 to cause the second nucleic acid 128 to be released from the compound carrier 124. In one or more illustrative examples, after being released from the compound carrier 124, at least a portion of the second nucleic acid 128 can be coupled to at least a portion of the first nucleic acid 118. In these scenarios, a portion of a first nucleotide sequence of the first nucleic acid 118 can be complementary to a portion of a second nucleotide sequence of the second nucleic acid 128. In at least some examples, the first nucleic acid 118 and the second nucleic acid 128 can be combined to form a double stranded nucleic acid that includes two strands that include at least a portion of the first nucleotide sequence of the first nucleic acid 118 and at least a portion of the second nucleotide sequence of the second nucleic acid 128. At least one of the strands of the double stranded nucleic acid formed from the first nucleic acid 118 and the second nucleic acid 128 can be collected and analyzed to determine that the compound 126 was present in a location of the array of sites 108 that corresponds to the site 110. As a result, the electrical responses produced by applying electrical signals to the liquid 120 in the presence of the compounds 126 and the biological cell 122 can be identified and analyzed.

In one or more illustrative examples, the compound 126 can include a candidate molecule for treating one or more biological conditions. In various examples, the one or more biological conditions can include diseases related to the central nervous system. In at least some examples, the compound 126 can be a small molecule that can include a candidate treatment for one or more biological conditions related to the human brain. In still other examples, the compound 126 can be a small molecule that can include a candidate treatment for one or more biological conditions related to the human heart. Measuring changes in electrical properties of the liquid 120 in response to the one or more biological cells 122 being exposed to the compound 126 can indicate a response of the one or more biological cells 122 to treatments of a biological condition that involves the compound 126. In at least some examples, measuring changes in electrical properties of the liquid 120 in response to the biological cell 122 being exposed to the compound 126 can indicate an effectiveness of the compound 126 in treating a biological condition.

Although the illustrative example of FIG. 1 describes features of a single semiconductor device 102, in one or more additional implementations, multiple semiconductor devices 102 can be disposed on substrate. For example, multiple semiconductor devices 102 can be disposed on a plate. To illustrate, a plate having a number of wells can be provided with a semiconductor device 102 disposed in individual wells. In one or more examples, the plate can have from 6 wells to 1536 wells or more arranged in an array. The plate can be coupled with a fluid delivery system having one or more inlet ports to provide liquid to the wells of the plate and one or more outlet ports to drain liquid from the plate. In various examples, patch clamp measurements can be performed in relation to semiconductor devices 102 located in individual wells of the plate. In one or more examples, at least a portion of the individual semiconductor devices 102 can perform patch clamp measurements with respect to liquids that include different therapeutic compounds.

Additionally, in one or more implementations, the semiconductor devices 102 described herein can be produced by one or more processes described in relation to and include one or more features described in relation to the semiconductor devices described in U.S. patent application Ser. No. 18/769,215, filed Jul. 10, 2024, and entitled “Semiconductor Devices To Measure Electrical Signals Of Material Disposed In Fluid,” which is incorporated by reference herein in its entirety.

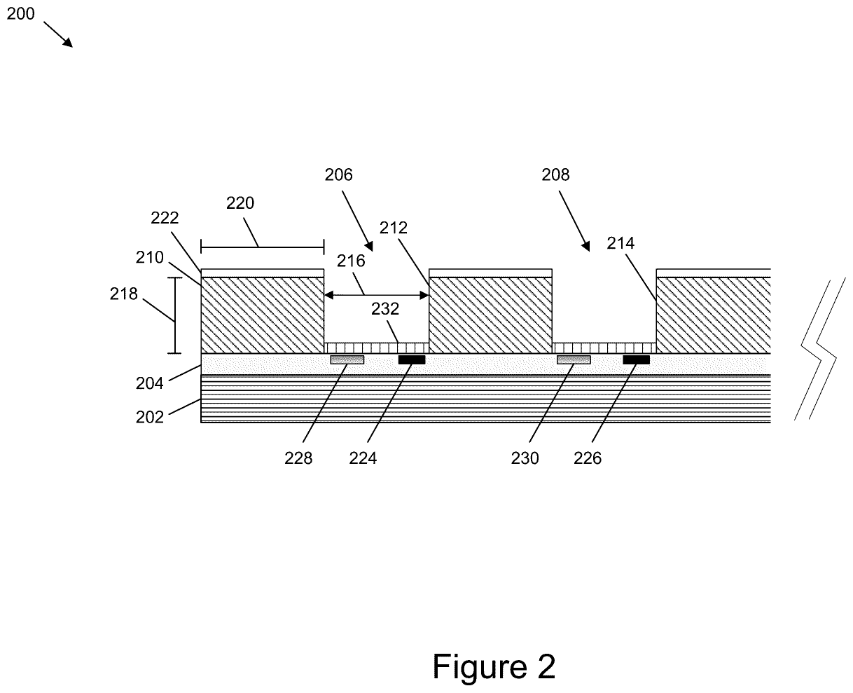

FIG. 2 is a diagram depicting a cross-section of at least a portion of an example semiconductor device 200 in which at least one of current or voltage can be applied to substances disposed in liquid, in accordance with one or more example implementations. In at least some examples, the first example semiconductor device 200 can correspond to an implementation of the semiconductor device 102 described in relation to FIG. 1.

The semiconductor device 200 can include a support structure 202. In at least some examples, the support structure 202 can be comprised of one or more substrates. The support structure 202 can also include one or more additional layers disposed on the one or more substrate. The support structure 202 can be comprised of one or more glass materials. The support structure 202 can also be comprised of one or more silicon wafers. For example, the support structure 202 can be comprised at least primarily of silicon. In one or more additional examples, the support structure 202 can be comprised of at least one of silicon carbide or silicon nitride. In one or more further examples, the support structure 202 can be comprised of polycrystalline silicon. In one or more illustrative examples, the support structure 202 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon.

Although not specifically shown in the illustrative example of FIG. 2, the semiconductor device 200 can include one or more additional layers disposed on and/or within the support structure 202. For example, the semiconductor device 200 can include a functional layer disposed at least one of on or within the support structure 202. The functional layer can include circuitry formed at least one of on or within the support structure 202. In one or more examples, the functional layer can include circuitry to apply at least one of voltage or current to liquid disposed on the semiconductor device 200 and to measure signals produced in response to the electrical stimulation provided to the liquid. In one or more illustrative examples, the functional layer can include circuitry to perform at least one of voltage clamp operations, current clamp operations, or dynamic clamp operations. In various examples, the functional layer can include analog-to-digital converters (ADCs), digital-to-analog converters (DACs), amplifiers, digital signal processing circuitry, one or more combinations thereof, and the like. The functional layer can also include routing circuitry to carry electrical signals to locations on a die and/or on packaging of the semiconductor device 200 for further processing. The circuitry of the functional layer can be produced using CMOS technologies.

Further, the semiconductor device 200 can include one or more passivation layers disposed at least one of on or within the support structure 202. The one or more passivation layers can be comprised of one or more oxide materials and/or one or more nitride materials. For example, the one or more passivation layers can be comprised of at least one of silicon oxide, silicon nitride, aluminum oxide, or titanium oxide. In one or more additional examples, the one or more passivation layers can be comprised of polycrystalline silicon. In various examples, the one or more passivation layers can be comprised of polycrystalline silicon doped with at least one of arsenic, phosphorus, or boron. The one or more passivation layers can also be comprised of undoped polycrystalline silicon. The one or more passivation layers can have thickness from about 0.5 micrometers (μm) to about 25 μm, from about 1 μm to about 20 μm, from about 2 μm to about 15 μm, from about 1 μm to about 10 μm, from about 5 μm to about 15 μm, from about 10 μm to about 20 μm, from about 1 μm to about 5 μm, from about 5 μm to about 10 μm, 10 μm to about 15 μm, from about 2 μm to about 8 μm, or from about 4 μm to about 10 μm.

The semiconductor device 200 can also include one or more thermal insulating layers 204. In one or more examples, the one or more thermal insulating layers 204 can be comprised of one or more polymeric materials. For example, the one or more thermal insulating layers 204 can be comprised of at least one of one or more epoxy-containing materials, one or more polyester-containing materials, one or more polypropylene-containing materials, one or more polyethylene-containing materials, one or more polyimide-containing materials, one or more acrylic-containing materials, or one or more combinations thereof. In one or more additional examples, the one or more thermal insulating layers 204 can be comprised of one or more mica-containing materials. In one or more further examples, the one or more thermal insulating layers 204 can be comprised of one or more ceramic materials. The one or more thermal insulating layers 204 can have thickness from about 0.1 micrometers (μm) to about 25 μm, from about 0.5 μm to about 20 μm, from about 0.1 μm to about 15 μm, from about 1 μm to about 10 μm, from about 5 μm to about 15 μm, from about 10 μm to about 20 μm, from about 1 μm to about 5 μm, from about 5 μm to about 10 μm, 10 μm to about 15 μm, from about 2 μm to about 8 μm, or from about 4 μm to about 10 μm.

The semiconductor device 200 can also include a number of sites. The number of sites can be structured to apply electrical stimulation to a discrete amount of liquid located in individual sites. In the illustrative example of FIG. 2, the semiconductor device 200 includes a first site 206 and a second site 208. Although the illustrative example of FIG. 2 shows two sites of the semiconductor device 200, the semiconductor device 200 can include a greater number of sites. For example, the semiconductor device 200 can include at least 100 sites, at least 500 sites, at least 1000 sites, at least 5000 sites, at least 10,000 sites, at least 25,000 sites, at least 50,000 sites, at least 100,000 sites, or more.

The sites 206, 208 can be defined by walls. The first site 206 can be enclosed by at least a first wall 210 and a second wall 212. In addition, the second site 208 can be enclosed by at least the second wall 212 and a third wall 214. Although not shown in the illustrative cross sectional view of FIG. 2, the sites 206, 208 can also be defined by walls disposed in rows perpendicular to the walls 210, 212, 214. In this way, the sites 206, 208 can be defined by a rectangular shape having boundaries formed by four walls. The sites 206, 208 can have an inner width 216 from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

In one or more examples, the walls 210, 212, 214 can be comprised of one or more hydrophobic materials and/or one or more low surface energy materials. In at least some examples, the walls 210, 212, 214 can be comprised of one or more polymeric materials. In one or more additional examples, the walls 210, 212, 214 can be comprised of one or more photoresist materials. In one or more illustrative examples, the walls 210, 212, 214 can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. In one or more further examples, the walls 210, 212, 214 can be comprised of a polysilicon-containing material. In still other examples, the walls 210, 212, 214 can be comprised of one or more silicon-containing materials. In one or more illustrative examples, the walls 210, 212, 214 can have a height 218 from about 1 nm to about 500 μm, from about 10 nm to about 400 μm, from about 100 nm to about 300 μm, from about 1 μm to about 200 μm, from about 5 μm to about 100 μm, from about 10 μm to about 50 μm, from about 50 μm to about 100 μm, from about 1 μm to about 10 μm, from about 10 μm to about 25 μm, from about 50 μm to about 150 μm, or from about 100 μm to about 200 μm. In one or more additional illustrative examples, the walls 210, 212, 214 can have a thickness 220 from about 0.1 μm to about 50 μm, from about 1 μm to about 30 μm, from about 5 μm to about 20 μm, from about 0.1 μm to about 1 μm, from about 0.5 μm to about 2 μm, from about 1 μm to about 5 μm, from about 1 μm to about 10 μm, from about 5 μm to about 15 μm, or from about 5 μm to about 10 μm.

The walls 210, 212, 214 can be covered by a coating 222. The coating 222 can be comprised of one or more layers of one or more hydrophilic materials. In one or more examples, the coating 222 can be comprised of one or more polymeric materials having hydrophilic properties. In one or more illustrative examples, the coating 222 can be comprised of a polyethylene glycol. In one or more additional illustrative examples, the coating 222 can be comprised of a polyvinyl alcohol. In one or more further illustrative examples, the coating 222 can be comprised of a poly(2-hydroxyethyl methacrylate) (PHEMA). The coating 222 can have a thickness from about 5 nanometers (nm) to about 25 μm, from about 20 nm to about 10 μm, from about 50 nm to about 5 μm, from about 10 nm to about 100 nm, from about 100 nm to about 500 nm, from about 500 nm to about 1 μm, from about 1 μm to about 10 μm, from about 500 nm to about 5 μm, or from about 100 nm to about 1 μm.

The individual sites 206, 208 can each include an electrode. The electrodes included in the individual sites 206, 208 can be electrically connected to circuitry of a functional layer of the support structure 202. In one or more examples, the electrodes of the individual sites 206, 208 can be disposed in the insulating layer 204. In the illustrative example of FIG. 2, a first electrode 224 can be located in the insulating layer 204 with respect to the first site 206 and a second electrode 226 can be located in the insulating layer 204 with respect to the second site 208. In one or more examples, the first electrode 224 and the second electrode 226 can be used to apply at least one of voltage or current to liquid disposed in the respective sites 206, 208. Additionally, at least one of the first electrode 224 or the second electrode 226 can be used to measure signals produced in response to electrical stimulation being provided to liquid disposed in the respective sites 206, 208. The electrodes 224, 226 can be comprised of one or more metallic materials. To illustrate, the electrodes 224, 226 can be comprised of at least one of sliver chloride, platinum, alloys of platinum, gold, or alloys of gold. In at least some examples, the electrodes 224, 226 can be placed in locations of the respective sites 206, 208 that correspond to locations of biological cells that can be present in the sites 206, 208.

In various examples, the individual sites 206, 208 can each include a heating element. The heating elements included in the individual sites can be electrically connected to circuitry of a functional layer of the support structure 202. In one or more examples, the heating elements of the individual sites 206, 208 can be disposed in the insulating layer 204. In the illustrative example of FIG. 2, a first heating element 228 can be located in the insulating layer 204 with respect to the first site 206 and a second heating element 230 can be located in the insulating layer 204 with respect to the second site 208. The heating elements 228, 230 can be comprised of one or more metallic materials. In at least some examples, the heating elements 228, 230 can be comprised of one or more wires. In one or more illustrative examples, the heating elements 228, 230 can be comprised of at least one of iron, chromium, copper, aluminum, nickel, or molybdenum. In one or more additional illustrative examples, the heating elements 228, 230 can be comprised of at least one of one or more ceramic materials, one or more silicon-containing materials, or one or more silicon carbide-containing materials. In at least some examples, the heating elements 228, 230 can be placed in locations of the respective sites 206, 208 that correspond to locations of nucleic acids coupled to a surface of the sites 206, 208. In one or more further illustrative examples, the heating elements 228, 230 can be placed to apply heat to the nucleic acids to cause the nucleic acids to be released from a surface of the sites 206, 208.

A coating layer 232 can be disposed within the individual sites 206. 208. The coating layer 232 can be comprised of one or more materials. In at least some examples, the coating layer 232 can be comprised of multiple materials. In one or more examples, the coating layer 232 can be comprised of materials that attract one or more types of molecules that are disposed within the sites 206, 208. For example, the coating layer 232 can be comprised of one or more first materials to attract biological cells to a first location of the sites 206, 208. In one or more additional examples, the coating layer 232 can be comprised of one or more second materials to attract nucleic acids to a second location of the sites 206, 208. In one or more further examples, the coating layer 232 can be comprised of one or more third materials to attract substrates coupled to compounds and nucleic acid molecules to a third location of the sites 206, 208. In various examples, the coating layer 232 can be comprised of one or more hydrophilic materials with one or more additional sections of at least one of the one or more first materials, the one or more second materials, or the one or more third materials disposed within the hydrophilic material. In this way, the coating layer 232 can include one or more islands within the hydrophilic material comprised of at least one of the one or more first materials, the one or more second materials, or the one or more third materials. In one or more illustrative examples, the coating layer 232 can be comprised of one or more polymeric materials. To illustrate, at least a portion of the coating layer 232 can be comprised of at least one of polyethylene glycol, polyvinyl alcohol, or poly(2-hydroxyethyl methacrylate) (PHEMA).

In one or more examples, liquid can be disposed in the sites 206, 208. For example, the walls 210, 212, 214 and the coating layer 232 can comprise wells that can store a volume of liquid. For example, the walls 210, 212, 214 can form side surfaces of the wells and the coating layer 232 can comprise a floor of the wells. In one or more illustrative examples, volumes of liquid disposed in the individual sites 206, 208 can include from about 1 picoliter (pL) to about 100 nanoliters (nL), from about 5 pL to about 10 nL, from about 10 pL to about 1 nL from about 1 pL to about 100 pL, from about 1 pL to about 50 pL, from about 1 pL to about 10 pL, from about 10 pL to about 500 pL, from about 10 pL to about 100 pL, from about 10 pL to about 50 pL, from about 50 pL to about 500 pL, from about 50 pL to about 250 pL, from about 100 pL to about 1 nL, from about 100 pL to about 500 pL, from about 500 pL to about 5 nL, from about 500 pL to about 2 nL, from about 500 pL to about 1 nL, from about 1 nL to about 100 nL, from about 1 nL to about 50 nL, or from about 1 nL to about 10 nL.

In at least some examples, the amount of liquid dispensed into the sites 206, 208 can comprise an aqueous solution having one or more biological cells. In various examples, the amount of liquid can form a droplet in the individual sites 206, 208. For example, in scenarios where the walls 210, 212, 214 are comprised of hydrophobic materials and the coating layer 232 is comprised of a hydrophilic material, the amount of liquid dispensed into the sites 206, 208 can bead into droplets on the hydrophilic portions of the site 206, 208 and move away from the walls 210, 212, 214. In this way, the amount of liquid in the sites 206, 208 can be electrically and fluidically isolated from each other and from amounts of liquid disposed in other sites of the semiconductor device 200. Accordingly, independent electrophysiological measurements can be produced for individual sites of the semiconductor device 200.

In one or more illustrative examples, fluid disposed within the sites 206, 208 can include biological cells, one or more therapeutic compounds, one or more additional reagents, one or more combinations thereof, and the like. In at least some examples, the fluid disposed within the sites 206, 208 can include at least one of NaCl, KCl, HEPES buffer, MgCl₂, CaCl₂, glucose, or ligands. In one or more additional examples, an immiscible fluid, such as oil, can be disposed above an aqueous liquid layer that is disposed in the sites 206, 208. The immiscible liquid can also serve as a separator of one or more volumes of liquid including biological cells.

In various examples, the semiconductor device 200 can be manufactured using one or more CMOS related manufacturing technologies. For example, the one or more thermal insulating layers 204 can be deposited on the support structure 202 using one or more material deposition techniques. Additionally, the walls 210, 212, 214 can be formed using one or more lithographic processes to pattern and etch one or more materials according to the location of the walls 210, 212, 214. Further, the coating 222 for the walls 210, 212, 214 can be formed using one or more lithographic processes to pattern and etch one or more materials comprising the coating 222 onto the walls 210, 212, 214. In still other examples, the coating layer 232 can be at least one of deposited using one or more lithographic techniques or implanted and grown on the one or more thermal insulating layers 204 and between the walls 210, 212, 214. In one or more illustrative examples, at least one of the electrodes 224, 226 or the heating elements 226, 228 can be formed using one or more metallic materials using at least one of one or more electroplating processes, one or more chemical vapor deposition processes, or one or more physical vapor deposition processes. In at least some examples, the electrodes 224, 226 and/or the heating elements 226, 228 can be formed according to one or more patterning and etching processes that correspond to a pattern and location of the electrodes 224, 226 and/or the heating elements 226, 228.

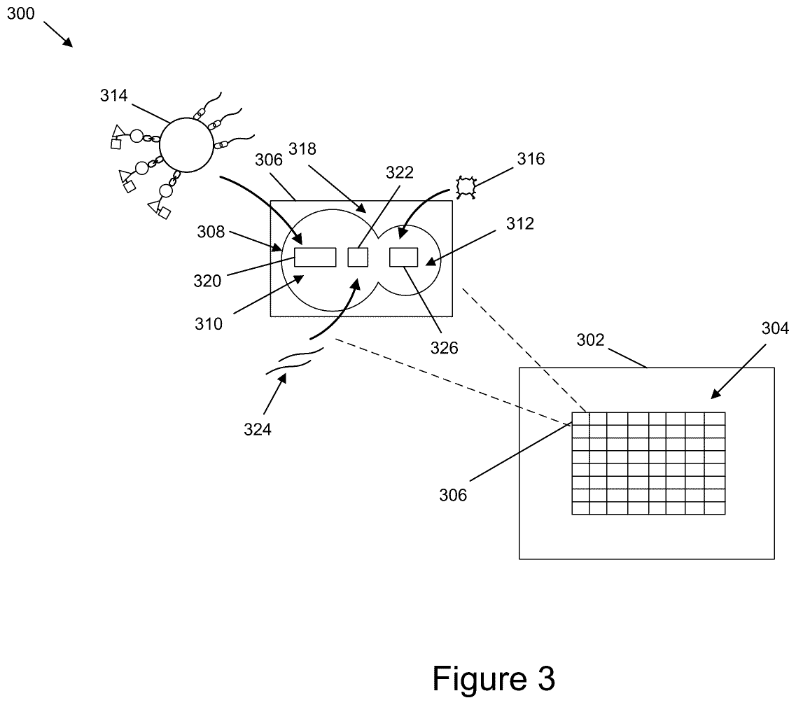

FIG. 3 is a diagram depicting a top view of a pattern of sites of a semiconductor device 300 in which electrical signals are applied to substances disposed in the sites, in accordance with one or more example implementations. In at least some examples, the example semiconductor device 300 can correspond to at least one of one or more implementations of the semiconductor device 102 described in relation to FIG. 1 or one or more implementations of the semiconductor device 200 described in relation to FIG. 2.

The semiconductor device 300 can be comprised of a substrate 302 that is comprised of one or more silicon-containing materials. For example, the substrate 302 can be comprised of at least one of silicon, silicon nitride, or polycrystalline silicon. The substrate 302 can include a die, also referred to herein as a chip, that includes circuitry to apply at least one of voltage or current to liquid disposed in or disposed on the die. The die can also include circuitry to measure signals produced in response to applying at least one voltage or current to the liquid. In various examples, biological material can be disposed in the liquid. In these scenarios, the signals produced in response to applying voltage and/or current to the liquid can indicate one or more characteristics of the biological material.

The semiconductor device 300 can include an array of sites 304. The array of sites 304 can be formed on and/or can be formed by the substrate 302. Individual sites of the array of sites 304 can include components to apply at least one of voltage or current to fluid disposed in the individual site and to measure signals produced in response to the voltage and/or current applied to the fluid. In one or more examples, the sites of the array of sites 304 can be arranged in rows and columns.

At least a portion of the individual sites of the array of sites 304 can be formed according to a pattern. The illustrative example of FIG. 3 includes a site 306 having a portion formed according to a pattern 308. In at least some examples, the pattern 308 can be from one or more hydrophilic materials disposed on a base surface or floor of the site 306. The pattern 308 can include a first section 310 and a second section 312. In one or more examples, the first section 310 and the second section 312 can each have a substantially circular shape. In various examples, the first section 310 and the second section 312 can comprise a continuous segment of the site 306. In one or more illustrative examples, a first surface area of the first section 310 can be larger than a second surface area of the second section 312.

The pattern 308 including the first section 310 and the second section 312 can be designed such that a single bead 314 can be disposed in the first section 310 of the individual site 306 and that the bead 314 is unable to be disposed in the second section 312 of the individual site 306. In one or more examples, the pattern 308 can be designed such that one or more biological cells 316 are disposed in the second section 312. In various examples, the first section 310 can have a diameter that is no greater than 2 times a diameter of the bead 314, no greater than 1.9 times a diameter of the bead 314, no greater than 1.8 times a diameter of the bead 314, no greater than 1.7 times a diameter of the bead 314, no greater than 1.6 times a diameter of the bead 314, or no greater than 1.5 times a diameter of the bead 314. Additionally, a diameter of the first section 310 can be at least 1.1 times a diameter of the bead 314, at least 1.2 times a diameter of the bead 314, at least 1.3 times a diameter of the bead 314, or at least 1.4 times a diameter of the bead 314. In one or more illustrative examples, a diameter of the first section 310 can be from 1.1 times to 2 times a diameter of the bead 314, from 1.2 times to 1.9 times a diameter of the bead 314, from 1.3 times to 1.8 times a diameter of the bead 314, from 1.1 times to 1.5 times a diameter of the bead 314, or from 1.5 times to 2 times a diameter of the bead 314. In one or more further illustrative examples, the second section 312 can have a diameter that is no greater than 1 times a diameter of the bead 314, no greater than 0.9 times a diameter of the bead 314, no greater than 0.8 times a diameter of the bead 314, no greater than 0.7 times a diameter of the bead 314, no greater than 0.6 times a diameter of the bead 314, or no greater than 0.5 times a diameter of the bead 314.

In various examples, the pattern 308 can have a surface area from about 0.05 millimeters squared (mm²) to about 500 mm², from about 0.1 mm² to about 400 mm², from about 0.5 mm² to about 300 mm², from about 1 mm² to about 200 mm², from about 0.05 mm² to about 10 mm², from about 1 mm² to about 100 mm², from about 10 mm² to about 100 mm², from about 100 mm² to about 500 mm², from about 100 mm² to about 300 mm², or from about 200 mm² to about 400 mm².

A surface of the first section 310 and the second section 312 of the pattern 308 can be comprised of a first material. In addition, a peripheral section 318 of the site 306 can be comprised of a second material that is different from the first material. For example, the first material can include one or more hydrophilic materials and/or one or more high surface energy materials. In one or more illustrative examples, the first material can comprise at least one of one or more oxides, one or more nitrides, or one or more polymeric materials. To illustrate, the first material can comprise at least one of silicon oxide, silicon nitride, titanium oxide, or aluminum oxide. In one or more additional illustrative examples, the first material can be comprised of at least one of a polyethylene glycol, a polyvinyl alcohol, or a poly(2-hydroxyethyl methacrylate) (PHEMA). In one or more further illustrative examples, the first material can include a hydrophilic material that is not adhering and/or not attracting with respect to at least one of the bead 314, the biological cell 316, or one or more nucleic acids.

Additionally, the second material comprising the peripheral section 318 can include one or more hydrophobic materials and/or one or more low surface energy materials. For example, the second material can be comprised of one or more polymeric materials. In various examples, the second material can include one or more photoresist materials. To illustrate, the second material can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. In one or more additional examples, the second material can be comprised of a polysilicon-containing material. In still other examples, the second material can be comprised of one or more silicon-containing materials.

In at least some examples, a surface of the first section 310 and the second section 312 can be include a number of portions that are formed to cause one or more moieties to be attracted to the respective portions. In one or more examples, the first section 310 can include a first portion 320 that is formed to attract the bead 314. In one or more additional examples, the first section 310 can include a second portion 322 that is formed to attract one or more nucleic acids 324. In one or more further examples, the second section 312 can include a third portion 326 that is formed to attract one or more biological cells 316. In various examples, the first portion 320, the second portion 322, and the third portion 326 can be formed to attract the respective moieties 314, 324, 316 based on one or more materials that comprise the first portion 320, the second portion 322, and the third portion 326. In one or more illustrative examples, the bead 314 can be comprised of a magnetic material and one or more materials of the first portion 320 can also include one or more magnetic materials that cause the bead 314 to be attracted to the first portion 320 in the presence of a magnetic field. In one or more additional illustrative examples, one or more linker molecules can be coupled to the second portion 322 such that the nucleic acids 324 are attracted to second portion 322. To illustrate, one or more linker molecules coupled to the second portion 322 can include one or more functional groups that can form bonds with one or more functional groups of the nucleic acids 324. In one or more further examples, at least one of the first portion 320 or the third portion 326 can include at least one of structures or surface coatings that attract the bead 314 and/or the biological cells 316 by at least one of electrophoresis, dielectrophoresis, or bioconjugation. In at least some examples, bio-conjugation can include the use of biotin-streptavidin linkages to bind at least one of the bead 314 to the first portion 320 or the one or more biological cells 316 to the third portion 326.

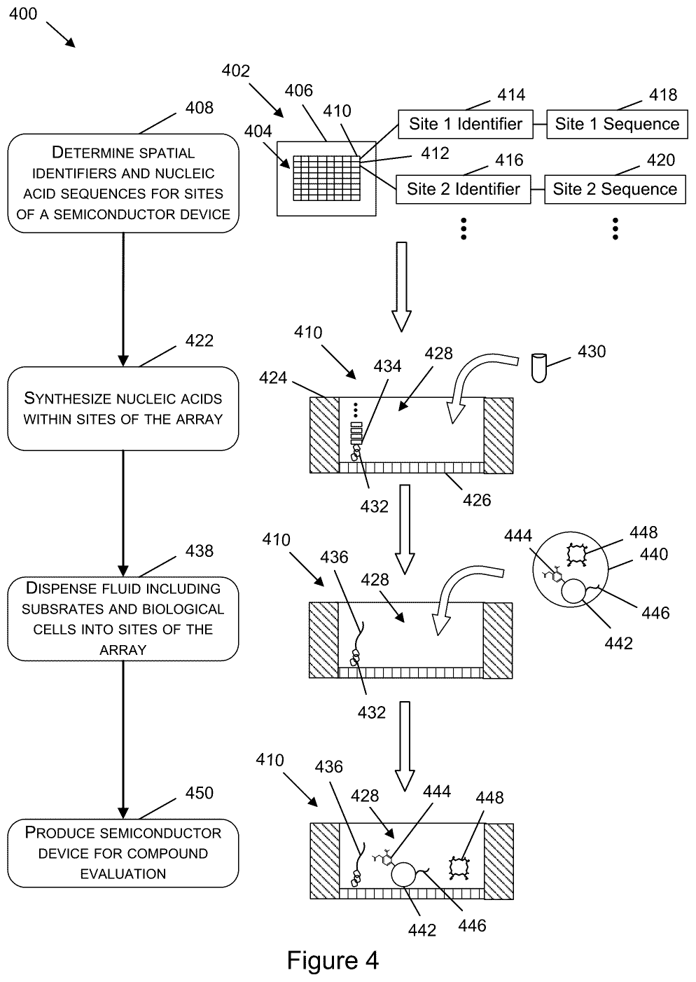

FIG. 4 is a diagram depicting a process 400 to prepare semiconductor device 402 including an array of sites 404 to test the electrical response of compounds disposed in individual sites, in accordance with one or more example implementations. The semiconductor device 402 can be comprised of a substrate 406 that is comprised of one or more silicon-containing materials. For example, the substrate 406 can be comprised of at least one of silicon, silicon nitride, or polycrystalline silicon. The substrate 406 can include a die, also referred to herein as a chip, that includes circuitry to apply at least one of voltage or current to liquid disposed in or disposed on the die. The die can also include circuitry to measure signals produced in response to applying at least one voltage or current to the liquid. In various examples, biological material can be disposed in the liquid. In these scenarios, the signals produced in response to applying voltage and/or current to the liquid can indicate one or more characteristics of the biological material. The array of sites 404 can be formed on and/or can be formed by the substrate 406. Individual sites of the array of sites 404 can include components to apply at least one of voltage or current to fluid disposed in the individual site and to measure signals produced in response to the voltage and/or current applied to the fluid. In one or more examples, the sites of the array of sites 404 can be arranged in rows and columns. In one or more illustrative examples, the semiconductor device 402 can correspond to at least one of one or more implementations of the semiconductor device 102 described in relation to FIG. 1, one or more implementations of the semiconductor device 200 described in relation to FIG. 2, or one or more implementations of the semiconductor device 300 described in relation to FIG. 3.

The process 400 can include, at 408, determining spatial identifiers and nucleic acid sequences for sites of the semiconductor device 402. For example, the array of sites 404 can include a number of individual sites, such as a first example site 410 and a second example site 412. In one or more examples, a first spatial identifier 414 corresponding to a location of the first site 410 within the array of sites 404 can be determined and a second spatial identifier 416 corresponding to a location of the second site 412 within the array of sites 404 can be determined. In at least some examples, the first spatial identifier 414 and the second spatial identifier 416 can include at least one of one or more symbols, one or more alphanumeric characters, or one or more words. In various examples, the first spatial identifier 414 can uniquely identify a location of the first site 410 within the array of sites 404 and the second spatial identifier 416 can uniquely identify a location of the second site 412 within the array of sites 404.

In addition, nucleotide sequences can be determined that correspond to the spatial identifiers of the individual sites of the array of sites 404. For example, a first nucleotide sequence 418 can be determined that corresponds to the first spatial identifier 414 and a second nucleotide sequence 420 can be determined that corresponds to the second spatial identifier 416. In one or more illustrative examples, the nucleotide sequences that correspond to the site identifiers of the sites of the array of sites 404 can be determined according to a process that corresponds to split-pool barcoding. In various examples, split-pool barcoding techniques can be modified such that rather than being able to trace a nucleotide sequence to a cell of origin, the nucleotide sequences derived with respect to the site identifiers can be generated to trace the individual nucleotide sequences to a given location of the array of sites 404. Examples of split pool barcoding techniques can be found in Kuijpers, L., Hornung, B., van den Hout-van Vroonhoven, M.C.G.N. et al. Split Pool Ligation-based Single-cell Transcriptome sequencing (SPLiT-seq) data processing pipeline comparison. BMC Genomics 25, 361 (2024) and Rosenberg A B, Roco C M, Muscat R A, Kuchina A, Sample P, Yao Z, Graybuck L T, Peeler D J, Mukherjee S, Chen W, Pun S H, Sellers D L, Tasic B, Seelig G. Single-cell profiling of the developing mouse brain and spinal cord with split-pool barcoding. Science. 2018 Apr. 13;360(6385):176-182. doi: 10.1126/science.aam8999. Epub 2018 Mar. 15. PMID: 29545511; PMCID: PMC7643870, which are incorporated by reference herein in their entirety. In one or more illustrative examples, at least 2 rounds, at least 3 rounds, at least 4 rounds, or at least 5 rounds of split pool barcoding can be performed to determine the nucleotide sequences that correspond to the spatial locations of the sites within the array of sites 404.

In one or more illustrative examples, individual nucleotide sequences that correspond to the spatial identifiers of the sites included in the array of sites can include from 10 nucleotides to 200 nucleotides, from 10 nucleotides to 100 nucleotides, from 10 nucleotides to 50 nucleotides, from 20 nucleotides to 80 nucleotides, from 50 nucleotides to 100 nucleotides, from 100 nucleotides to 200 nucleotides, or from 150 nucleotides to 200 nucleotides.

The process 400 can also include, at 422, synthesizing nucleic acids within sites of the array of sites 404 according to the nucleotide sequences that correspond to a given spatial identifier of the individual sites. In one or more examples, individual sites, such as the first site 410, can include walls 424 and a floor that is comprised of a coating layer 426. The walls 424 can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The coating layer 426 can be comprised of one or more hydrophilic materials. The walls 424 and the coating layer 426 can form a well 428 in which nucleic acids can be synthesized.

In one or more examples, nucleic acids can be synthesized within the well 428 according to an enzymatic process. In at least some examples, the enzymatic process for the synthesis of nucleic acids within the well 428 can be performed using one or more aqueous reaction solutions 430 that include the components for synthesizing the nucleic acids. In various examples, polynucleotide phosphorylase (PNPase) can be used to produce single stranded nucleic acid molecules by adding single nucleotides to a growing chain of nucleotides. Enzymatic processes based on PNPase to produce oligonucleotides can add modified nucleoside diphosphates to an oligonucleotide chain. The modified nucleoside diphosphates can have 3′ blocking groups that enable the addition of nucleotides to an oligonucleotide chain. Additionally, oligonucleotides can be synthesized with T4 RNA ligase (T4Rnl) using modified nucleoside diphosphates with a 3′ blocking group that are different from the modified nucleoside diphosphates used in PNPase synthesis processes to add nucleotides to an oligonucleotide chain. DNA polymerases can also be used to enzymatically form oligonucleotides. To illustrate, terminal deoxynucleotidyl transferase (TdT) can be used to add nucleotides to an oligonucleotide chain.

In at least some examples, nucleic acids can be synthesized by adding nucleotides to a molecular scaffold that comprises an intermediate oligonucleotide chain. For example, deoxyribonucleic acid (DNA) molecules and ribonucleic acid (RNA) molecules can be formed by coupling monomer units comprised of adenine (A), guanine (G), cytosine (C), and thymine (T), in the case of DNA, or A, G, C, and uracil (U), in the case of RNA. Typically, synthetic polynucleotides are produced according to a number of predetermined sequences. The predetermined sequences can correspond to at least one of the primers used in polynucleotide sequencing operations. The predetermined sequences can also correspond to identifiers, such as the spatial identifiers of the sites of the array of sites 404. In one or more illustrative examples, a nucleic acid can be synthesized within the well 428 having a predetermined sequence that includes, at least in part, the first spatial identifier 414 of the first site 410.

In various examples, one or more first linker molecules, such as a first linker molecule 432, can be coupled to the coating layer 426. The first linker molecule 432 can bind synthesized nucleic acids to the coating layer 426. The first linker molecule 432 can also be an initiator molecule by which a series of individual nucleotides 434 can be added to produce a nucleic acid within the well 428 according to a predetermined sequence associated with the first site 410. In one or more illustrative examples, the addition of individual nucleotides 434 to the first linker molecule 432 according to a predetermined nucleic acid sequence that includes at least the first spatial identifier 414 can include a stepwise process that includes a number of reaction cycles of an oligonucleotide synthesis process. The number of reaction cycles of the oligonucleotide synthesis process can correspond to a length of the predetermined nucleic acid sequences. The length of the predetermined nucleic acid sequence can correspond to the number of nucleotides in a chain of nucleotides. In one or more additional examples, the number of reaction cycles of the nucleotide addition process can correspond to a desired length of the synthesized oligonucleotides. Individual reaction cycles of the oligonucleotide synthesis process can include adding one or more aqueous reaction solutions 430 that include a number of nucleotide building blocks and one or more enzymes. The composition of the one or more reaction solutions can facilitate the addition of the nucleotide building blocks to a 3′-OH end of the first linker molecule 432. In one or more illustrative examples, the composition of the one or more reaction solutions can lower the pKa of 3′-OH groups at the ends of the first linker molecule 432 in preparation for the covalent joining of the 3′-OH end of the first linker molecule 432 with the 5′ phosphate moieties of dNTPs included in the one or more synthesis solutions. The joining of the 3′-OH end of the first linker molecule 432 with the 5′ phosphate moieties of dNTPs can be facilitated by the one or more enzymes included in the one or more reaction solutions. In one or more additional illustrative examples, the first linker molecule 432 can include an initiator molecule for synthesizing a nucleic acid having at least a portion of the nucleotide sequence of an M13 phage genome. In at least some examples, the first linker molecule 432 can be bound to the coating layer through electrostatic interactions. The electrostatic interactions can include at least one of ionic bonding interactions or van der Waals forces.

Each reaction cycle of adding one or more instances of a nucleotide to the intermediate nucleic acid can take place under a set of reaction conditions to facilitate the joining of one or more instances of a nucleotide to the intermediate oligonucleotide. The reaction conditions can include a duration for individual reaction cycles and one or more reaction temperatures. In one or more examples, the duration of an individual reaction cycle to add one or more instances of a nucleotide to intermediate oligonucleotides can be from about 30 seconds to about 10 minutes, from about 1 minute to about 8 minutes, from about 2 minutes to about 6 minutes, from about 1 minute to about 3 minutes, from about 2 minutes to about 4 minutes, or from about 3 minutes to about 5 minutes. In one or more additional examples, reaction temperatures for an individual reaction cycle of the nucleotide addition process can be from about 20° C. to about 45° C., from about 25° C. to about 40° C., from about 20° C. to about 30° C., from about 30° C. to about 40° C., or from about 35° C. to about 45° C. In various examples, the additional of one or more instances of a nucleotide to an intermediate oligonucleotide can be performed at atmospheric pressure.

Individual reaction cycles to add one or more instances of a nucleotide to intermediate oligonucleotides can be terminated by at least one of applying heat to the reaction mixture or adding a chelating agent. For example, individual reaction cycles of the nucleotide addition process can be terminated by heating the reaction mixture to temperatures from about 65° C. to about 100° C. for a duration from about 2 minutes to about 15 minutes. Additionally, individual reaction cycles of the nucleotide additional process can be terminated by adding ethylenediaminetetraacetic acid (EDTA) to the reaction mixture. In various examples, a final concentration of EDTA in the reaction mixture can be from about 20 millimolar (mM) to about 50 mM. Further, one or more washing solutions can be applied to the well 428 after the termination of an individual reaction cycle and before the start of a next reaction cycle that adds one or more instances of another nucleotide to the intermediate oligonucleotides. In one or more examples, the one or more washing solutions can include a buffer solution comprising at least one of one or more exonucleases or one or more phosphatases. In at least some examples, the one or more washing solutions can be heated for a period of time. In one or more illustrative examples, the one or more washing solutions can be heated at temperatures from about 30° C. to about 95° C. for a duration from about 5 minutes to about 40 minutes.

In one or more examples, a first reaction cycle can add one or more instances of a first nucleotide to at least a portion of the linker molecule 432 to produce a first intermediate oligonucleotide. Additional reaction cycles can be implemented to add other nucleotides to the first intermediate oligonucleotide until at least one reaction cycle has been performed in relation to the individual nucleotides of a predetermined nucleic acid sequence. To illustrate, a second reaction cycle can be performed to add one or more instances of a second nucleotide to the first intermediate oligonucleotide to produce a second intermediate oligonucleotide, a third reaction cycle can be performed to add one or more instances of a third nucleotide to the second intermediate oligonucleotide to produce a third intermediate oligonucleotide, and a fourth reaction cycle can be performed to add one or more instances of a fourth oligonucleotide to the third intermediate oligonucleotide to produce a fourth intermediate oligonucleotide. Additional reaction cycles can continue to be performed until completion of one or more predetermined nucleic acid sequence is reached.

In one or more additional examples, nucleic acids can be synthesized within the well 428 using light-directed oligonucleotide synthesis. In various examples, the coupling of nucleotides can include successively adding nucleotides to an intermediate oligonucleotide chain until a completed oligonucleotide is produced having a sequence of bases that corresponds to the predetermined sequence. In one or more instances, the addition of nucleotides can be controlled such that a given nucleotide is added to one or more specified intermediate oligonucleotide chains. For example, during one round of synthesizing oligonucleotides, the nucleotide adenine can be added to a number of intermediate oligonucleotide chains for which adenine is the next nucleotide in the predetermined sequence. The process can continue with another round of oligonucleotide synthesis causing thymine nucleotides to be added to a number of intermediate oligonucleotide chains for which thymine is the next nucleotide in the predetermined sequence. That is, nucleotides can be selectively added to intermediate oligonucleotide chains according to the predetermined oligonucleotide sequences.

In one or more examples, nucleotides can be selectively added to intermediate oligonucleotide sequences using protecting groups. Protecting groups can prevent new nucleotides from being added to intermediate oligonucleotide sequences by preventing reactions between reactive groups of free nucleotides and reactive groups of the intermediate oligonucleotide chains. In scenarios that involve the use of light-directed oligonucleotide synthesis, 5′ protecting groups that can be cleaved by the application of specified wavelengths of electromagnetic radiation can be added to oligonucleotide chains. In one or more illustrative examples, the wavelengths of electromagnetic radiation can correspond to ultraviolet radiation wavelengths. To illustrate, the electromagnetic radiation being applied can include wavelengths from about 10 nanometers to about 400 nanometers. The application of electromagnetic radiation to sites of the array of sites 404 can be controlled through the use of masking materials that are deposited according to a pattern. In at least some examples, the masking materials can include photolithographic masking materials. For a given cycle of the oligonucleotide synthesis process, the pattern can correspond to sites in which a nucleotide is to be added to an intermediate oligonucleotide sequence. In this way, the electromagnetic radiation can be applied to remove the protecting groups from the intermediate oligonucleotide chains in sites where the masking material is not present to add a nucleotide to the intermediate oligonucleotide. Further, for sites where the nucleotide is not to be added during the given cycle, the masking material may prevent the electromagnetic radiation from contacting the protecting group and the protecting group can remain coupled to the intermediate oligonucleotides present in those sites. In still other examples, light-directed synthesis of oligonucleotides can be performed using a number of micromirrors to direct electromagnetic radiation to a pattern of sites.

Examples of light directed oligonucleotide synthesis can be found in Fodor S P, Read J L, Pirrung M C, Stryer L, Lu A T, Solas D. Light-directed, spatially addressable parallel chemical synthesis. Science. 1991 Feb. 15;251(4995): 767-73. doi: 10.1126/science.1990438. PMID: 1990438; Pease A C, Solas D, Sullivan E J, Cronin M T, Holmes C P, Fodor S P. Light-generated oligonucleotide arrays for rapid DNA sequence analysis. Proc Natl Acad Sci U S A. 1994 May 24;91(11): 5022-6. doi: 10.1073/pnas.91.11.5022. PMID: 8197176; PMCID: PMC43922; Jory Lietard, Adrien Leger, Yaniv Erlich, Norah Sadowski, Winston Timp, Mark M Somoza, Chemical and photochemical error rates in light-directed synthesis of complex DNA libraries, Nucleic Acids Research, Volume 49, Issue 12, 9 Jul. 2021, Pages 6687-6701; and Singh-Gasson, S., Green, R., Yue, Y. et al. Maskless fabrication of light-directed oligonucleotide microarrays using a digital micromirror array. Nat Biotechnol 17, 974-978 (1999), which are incorporated by reference herein in their entirety.

After synthesizing one or more first nucleic acids 436 within the wells 428, at 438, the process 400 can include dispensing fluid 440 including beads 442 and biological cells 448 into sites 410 of the array of sites 404. In one or more examples, the fluid 440 can be dispensed into the sites 410 in discrete volumes. For example, droplets can be supplied to individual sites 410 of the array of sites 404. In one or more additional examples, the fluid 440 can be dispensed into the sites 410 in one or more batches. In one or more further examples, the fluid 440 can be dispensed into the sites 410 in one or more continuous or semi-continuous streams. In one or more illustrative examples, the fluid 440 can include one or more buffer solutions suitable for containing nucleic acids and therapeutic compounds. To illustrate, the fluid 440 can include an aqueous solution having at least one of tris(hydroxymethyl)aminomethane(Tris), ethylenediaminetetraacetic acid (EDTA), one or more salts, or one or more surfactants. In various examples, the fluid 440 can also include at least one of sodium chloride, potassium chloride, magnesium chloride, sodium dodecyl sulfate (SDS), or Triton-X100.

The fluid 440 can include a bead 442. In one or more illustrative examples, a number of beads 442 included in the fluid 440 can correspond to a number of sites 410 present in the array of sites 404. For example, number of beads 442 included in the fluid 440 can be from 0.8 times to 1.2 times, from 0.9 times to 1.1 times, or no greater than 1 times the number of sites 410 included in the array of sites 404. One or more compounds 444 can be coupled to the bead 442. The one or more compounds 444 can have an average molecular weight that is no greater than about 10,000 grams/mol (g/mol), no greater than 5000 g/mol, no greater than 2500 g/mol, no greater than 1000 g/mol, or no greater than 500 g/mol.

In one or more examples, the compound 444 can be coupled to the bead 442 using a second linker molecule. The compound 444 can be included in the fluid 440 such that electrical testing can be performed using the semiconductor device 402 to determine whether or not the compound 444 impacts one or more features of the biological cells 448. Additionally, one or more second nucleic acids 446 can be coupled to the bead 442. At least a portion of the nucleotide sequence of the one or more second nucleic acids 446 can include an identifier of the compound 444. In various examples, the one or more second nucleic acids 446 can be coupled to the bead 442 by a third linker molecule. The one or more second nucleic acids 446 can be formed on the bead 442 using one or more implementations of the enzymatic nucleotide synthesis process described with respect to synthesizing the one or more first nucleic acids 436.

In one or more illustrative examples, the first linker molecule 432 can bind the first nucleic acid 436 by a first type of interaction, the second linker molecule can bind the compound 444 by a second type of interaction, and the third linker molecule can bind the second nucleic acid 446 by a third type of interaction. In at least some examples, at least one of the first type of interaction, the second type of interaction, or the third type of interaction can differ from at least one other of the first type of interaction, the second type of interaction, or the third type of interaction. In still other examples, at least one of the first type of interaction, the second type of interaction, or the third type of interaction can be the same as at least one other of the first type of interaction, the second type of interaction, or the third type of interaction. The first type of binding interaction, the second type of binding interaction, and the third type of binding interaction can include at least one of a photocleavable interaction, a thermally cleavable interaction, an enzymatically cleavable interaction, or a disulfide interaction.

The process 400 can include, at 450, producing a semiconductor device 402 that can be used for evaluating a number of compounds. For example, the process 400 can be implemented to produce a semiconductor device 402 having hundreds of sites, thousands of sites, tens of thousands of sites, or more, that are the same as or similar to site 410. That is, the process 400 can be implemented to produce a semiconductor device 402 having one or more first nucleic acids 436 coupled to a coating layer 426 of the sites 410 and having a bead 442 and one or more biological cells including in the sites 410. In at least some examples, the one or more first nucleic acids 436 can be disposed in a first location within the sites 410, the bead 442 can be disposed in a second location within the sites 410, and the one or more biological cells 448 can be disposed in a third location within the sites 410. In various examples, the first location can be distinct from the second location and the third location and the second location can be distinct from the third location. After implementing the process 400, the semiconductor device 402 can be configured for evaluating hundreds, thousands, up to millions of compounds in relation to the biological cells 448 by applying electrical signals to the sites 410 and measuring the electrical responses to the signals. In one or more illustrative examples, after the one or more first nucleic acids 436 have been synthesized and fluid 440 including the beads 442 and the biological cells 448 has been disposed in the sites 410, a layer of a hydrophobic liquid, such as an oil can be disposed over the array of sites 408.

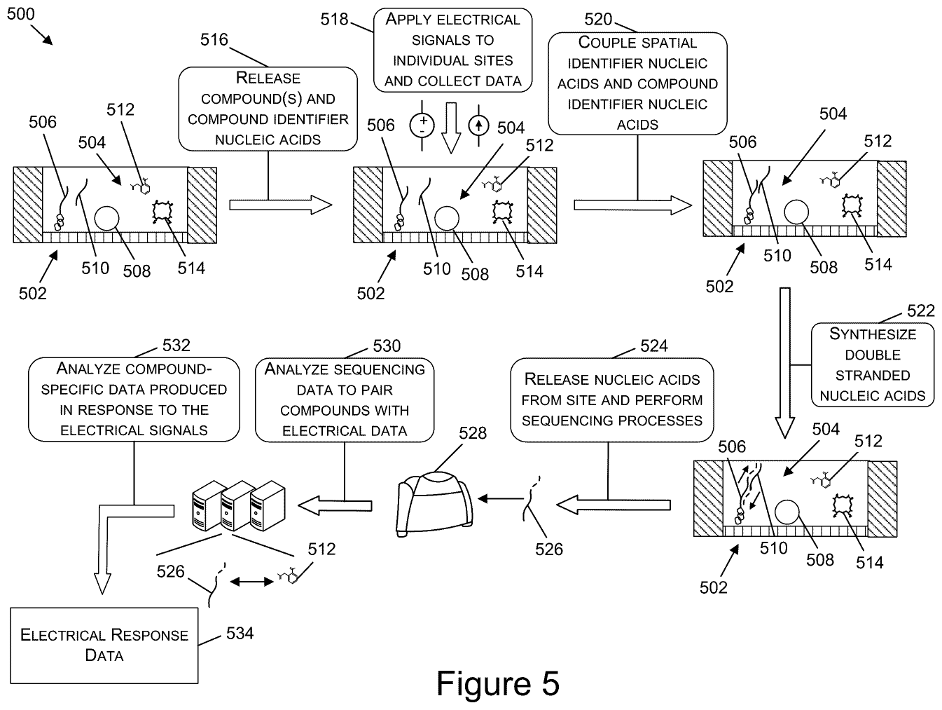

FIG. 5 is a diagram depicting a process 500 to track the electrical response of compounds disposed in sites of a semiconductor device, in accordance with one or more implementations. In one or more examples, the semiconductor device for which the process 500 is applied can correspond to the semiconductor device 402 that is produced according to one or more implementations of the process 400 described in relation to FIG. 4. For example, the semiconductor device used in the process 500 can include a number of sites 502 with individual sites 502 forming wells 504. The wells 504 can include spatial identifier nucleic acids 506 located in a first location of the wells 504. The spatial identifier nucleic acids 506 can include a nucleotide sequence with a portion of the nucleotide sequence indicating an identifier of the site 502 in which the spatial identifier nucleic acids 506 are located. In addition, beads 508 can be located in a second location of the wells 504 with the beads 508 having one or more compound identifier nucleic acids 510 and one or more compounds 512 bound to the beads 508. The one or more compound identifier nucleic acids 510 can include a nucleotide sequence with a portion of the nucleotide sequence indicating an identifier of the compound 512. Further, one or more biological cells 514 can be located in a third location of the wells 504. In at least some examples, an oil can be disposed over the sites 502 of the array of sites. To illustrate, a fluorinated oil can be dispensed over the array of sites. In one or more additional examples, the process 500 can be performed in a relatively high humidity environment such that individual sites 502 of the array of sites can be electrically isolated and to minimize evaporation of fluid disposed within the wells 504 of the individual sites 502. In still other examples, one or more images can be captured of the array of sites after the preparing individual sites 502 of the semiconductor device to include the one or more spatial identifier nucleic acids 506, the bead 508, the one or more compound identifier nucleic acids 510, one or more instances of the compound 512, and one or more biological cells 514.

The process 500 can include, at 516, releasing the one or more compounds 512 and the one or more compound identifier nucleic acids 510 from the beads 508. In one or more examples, the one or more compounds 512 can be released from the beads 508 by cleaving a linkage between the one or more compounds 512 and the beads 508. In one or more additional examples, the one or more compound identifier nucleic acids 510 can be released from the beads 508 by cleaving a linkage between the one or more compound identifier nucleic acids 510 and the beads 508. In various examples, the one or more linkage cleaving techniques can be applied to the sites 502 to cause the one or more compound identifier nucleic acids 510 to be released form the beads 508 and to cause the one or more compounds 512 to be released from the beads 508.

In one or more illustrative examples, a first linkage cleaving technique can be applied to the sites 502 to cause the one or more compound identifier nucleic acids 510 to be released from the beads and a second linkage cleaving technique can be applied to the sites 502 to cause the one or more compounds 512 to be released from the beads 508. In one or more additional illustrative examples, the first linkage cleaving technique can be different from the second linkage cleaving technique. In still other illustrative examples, the first linkage cleaving technique can be the same as the second linkage cleaving technique. The first linkage cleaving technique and the second linkage cleaving technique can include a light-based linkage cleaving technique, an enzyme based linkage cleaving technique, a temperature based linkage cleaving technique, or a chemical based linkage cleaving technique. In at least some examples, a chemical based linkage cleaving technique can include applying one or more reducing reagents to break disulfide bonds used to link at least one of the compound identifier nucleic acids 510 and/or the compounds 512 to the beads 508. In various examples, steric hindrance of the bead 508 can minimize or prevent interaction between the compound 512 and the one or more biological cells 514. In one or more examples, at least one of the compound identifier nucleic acid 510 or the compound 512 can be released from the bead 508 by applying electromagnetic radiation having wavelengths in the ultraviolet spectrum. For example, at least one of the compound identifier nucleic acid 510 or the compound 512 can be released from the bead 508 by application electromagnetic radiation having wavelengths from about 300 nanometers to about 400 nanometers.

At 518, the process 500 can include applying electrical signals to individual sites 502 and collecting data indicating the response to the electrical signals. In one or more examples, at least one of voltage signals or current signals can be applied to individual sites 502 using one or more electrodes disposed in the individual sites 502. The response to the electrical signals can also be measured by one or more electrodes present in the individual sites 502. In various examples, by releasing the compound 512 from the bead 508, the impact of the compound 512 on at least one of the function or structure of the biological cells 514 can be determined by measuring the response of one or more electrical characteristics of the fluid within the wells 504 of the individual sites 502. For example, changes in at least one of voltage or current can be measured from one or more times before releasing the compound 512 from the bead 508 and one or more times after releasing the compound 512 from the bead 508. In one or more illustrative examples, the electrical signals can be applied according to one or more patch clamp protocols that include applying a voltage protocol across different concentrations of the one or more compounds and analyzing an IV curve that corresponds to gate function measured at peak current values. In at least some cases, temporal aspects of the response signal can also be analyzed. Examples of patch clamp protocols that can be applied in relation to biological cells included in the sites of the patch clamp apparatus can include those described in Seibertz, F., Rapedius, M., Fakuade, F. E. et al. A modern automated patch-clamp approach for high throughput electrophysiology recordings in native cardiomyocytes. Commun Biol 5, 969 (2022), which is incorporated by reference herein in its entirety.

Although the illustrative example of FIG. 5 has described the process 500 as including the release of both the compound identifier nucleic acid 510 and the compound 512 from the bead 508 before applying the electrical signals, in other situations, the compound identifier nucleic acid 510 can be released from the bead 508 after the electrical signals have been applied to the individual sites 502. In these scenarios, the linkage techniques binding the compound identifier nucleic acid 510 and the compound 512 to the bead 508 can be different. That is, in instances where the compound 512 is released from the bead 508 by applying ultraviolet electromagnetic radiation to the site 502, the compound identifier nucleic acid 510 can be released from the bead 508 by applying one or more enzymes to the site 502, by applying heat to the site 502, or by applying one or more chemical reagents to the site 502.

The process 500 can also include, at 520, coupling the spatial identifier nucleic acid 506 with the compound identifier nucleic acid 510. In one or more examples, the spatial identifier nucleic acid 506 can be coupled with the compound identifier nucleic acid 510 by a portion of the spatial identifier nucleic acid 506 hybridizing with a portion of the compound identifier nucleic acid 510. To illustrate, a portion of the nucleotide sequence of the compound identifier nucleic acid 510 and be complementary with respect to a portion of the nucleotide sequence of the spatial identifier nucleic acid 506. That is, in situations where a position of the nucleotide sequence of the spatial identifier nucleic acid 506 or the compound identifier nucleic acid 510 includes an adenine molecule, a corresponding position in the nucleotide sequence of the other one of the spatial identifier nucleic acid 506 or the compound identifier nucleic acid 510 can include a thymine molecule. Additionally, in scenarios where a position of the nucleotide sequence of the spatial identifier nucleic acid 506 or the compound identifier nucleic acid 510 includes a cytosine molecule, a corresponding position in the nucleotide sequence of the other one of the spatial identifier nucleic acid 506 or the compound identifier nucleic acid 510 can include a guanine molecule. Further, in instances where a position of the nucleotide sequence of the spatial identifier nucleic acid 506 or the compound identifier nucleic acid 510 includes a thymine molecule, a corresponding position in the nucleotide sequence of the other one of the spatial identifier nucleic acid 506 or the compound identifier nucleic acid 510 can include an adenine molecule and in implementations where a position of the nucleotide sequence of the spatial identifier nucleic acid 506 or the compound identifier nucleic acid 510 includes a guanine molecule, a corresponding position in the nucleotide sequence of the other one of the spatial identifier nucleic acid 506 or the compound identifier nucleic acid 510 can include a cytosine molecule. In examples where the spatial identifier nucleic acid 506 and the compound identifier nucleic acid 510 are ribonucleic acids (RNA), thymine molecules of the spatial identifier nucleic acid 506 and the compound identifier nucleic acid 510 can be paired with uracil molecules of the other one or the spatial identifier nucleic acid 506 and the compound identifier nucleic acid 510. In one or more illustrative examples, the spatial identifier nucleic acid 506 can have a first nucleotide sequence including a first hybridization region and the compound identifier nucleic acid 510 can have a second nucleotide sequence including a second hybridization region that is complementary to the first hybridization region. In this way, the spatial identifier nucleic acid 506 can be coupled to the compound identifier nucleic acid 510 through hydrogen bonding between the complementary hybridization regions of the spatial identifier nucleic acid 506 and the compound identifier nucleic acid 510.

After hybridization between a portion of the nucleotide sequence of the spatial identifier nucleic acid 506 and a complementary portion of the nucleotide sequence of the compound identifier nucleic acid 510, the array of sites can be washed using one or more buffer solutions. In one or more examples, unbound nucleic acids and/or loosely bound nucleic acids can be removed from the array of sites by the rinsing with the one or more buffer solutions.

At 522, the process 500 can include synthesizing one or more double stranded nucleic acids. In one or more examples, one or more solutions including DNA polymerase, primers and deoxynucleotide triphosphates (dNTPs) can be provided to the wells 504 of the sites 502 included in an array of sites of a semiconductor device. After adding the components to be used for synthesizing the double stranded nucleic acids, an oil can be applied to the array of sites. For example, a fluorinated oil can be applied to the array of sites. In one or more additional examples, the operation 522 can be performed in a relatively high humidity environment. By capping the sites 502 with an oil or by performing the operation 522 in a relatively high humidity environment, evaporation of fluid disposed in the individual sites 502 can be minimized and the components used to synthesize the double stranded nucleic acids can remain in the wells 504 during the synthesis process. The double stranded nucleic acid synthesis process can cause the nucleotide sequence of the spatial identifier nucleic acid 506 to be expanded with the expanded portion being complementary to the nucleotide sequence of the compound identifier nucleic acid 510. Additionally, the double stranded nucleic acid synthesis process can cause the nucleotide sequence of the compound identifier nucleic acid 510 to be expanded with the expanded portion being complementary to the nucleotide sequence of the spatial identifier nucleic acid 506. In this way, each strand of the double stranded nucleic acid includes information indicating the spatial location of the individual site 502 in which the double stranded nucleic acid is synthesized and indicating the compound that is present in the individual site 502.

Additionally, after expanding the nucleotide sequences of the spatial identifier nucleic acid 506 and the compound identifier nucleic acid 510, the sites 502 of the semiconductor device can undergo an additional rinsing process with one or more buffer solutions. One or more additional solutions can then be added to the wells 504 of the semiconductor device that include DNA ligase and adenosine triphosphate. The DNA ligase and adenosine triphosphate can be used to finalize the synthesis of the double stranded nucleic acid molecules within the wells 504. For example, the DNA ligase can complete any gaps on the sticky ends of the strands to complete the double stranded nucleic acids. In various examples, at least one of oil or a high humidity environment can be applied to the semiconductor device after delivering the ligation reaction components to the wells 504 to minimize evaporation of fluid disposed in the individual sites 502 and to cause the components used in the ligation reaction to remain in the wells 504 during the ligation process.

Further, the process 500 can include, at 524, releasing at least one strand of the double stranded nucleic acid from the site 502 and performing one or more sequencing processes. For example, heat can be applied to the site 502 to cause the temperature within the well 504 to increase above a denaturation temperature of the double stranded nucleic acid. To illustrate, the site 502 can be heated using one or more heating elements within a floor of the site to cause a temperature within the well 504 to be from about 80° C. to about 100° C. By increasing the temperature in the well 504 to be above the denaturation temperature of the double stranded nucleic acid, a strand 526 can decouple from the double stranded nucleic acid and be collected. In one or more additional examples, the individual strands 526 can be released from the double stranded using other techniques. In one or more illustrative examples, one or more enzymes can be supplied to the sites 502 to cause the individual strands 526 to be released from the double stranded nucleic acids formed in the wells 504. In one or more additional illustrative examples, one or more solutions including one or more reducing reagents or one or more acidic components can be applied to the sites 502 to cause the individual strands 526 to be released from the double stranded nucleic acids synthesized in the wells 504. In scenarios where an oil is present on the array of sites during the ligation process, the oil and any remaining components from the ligation reaction can be removed from the array of sites prior to releasing the individual strands 526 from the double stranded nucleic acids present in the wells 504.

The collected individual nucleic acid strands 526 can then be provided to one or more sequencing machines 528. The one or more sequencing machines 528 can implement one or more polymerase chain reaction processes to cause amplification of the individual nucleic acid strands 526. In various examples, amplification of the individual nucleic acid strands 526 can include cycles of denaturation, annealing and extension, resulting from thermocycling. Other amplification methods can include the ligase chain reaction, strand displacement amplification, nucleic acid sequence-based amplification, and self-sustained sequence-based replication.

The one or more sequencing machines 528 can also generate sequencing data that includes sequencing reads that indicate the nucleotide sequences of the individual nucleic acid strands 526. In one or more illustrative examples, the one or more sequencing machines 528 can implement one or more next generation sequencing processes with respect to the individual nucleic acid strands 526. Next generation sequencing can include sequencing technologies having increased throughput as compared to traditional Sanger-and capillary electrophoresis-based approaches, for example, with the ability to generate hundreds of thousands of relatively small sequencing reads at a time. Some examples of next generation sequencing techniques include, but are not limited to, sequencing by synthesis, sequencing by ligation, and sequencing by hybridization.

At 530, the process 500 can include analyzing the sequencing data to pair compounds with electrical data produced with respect to individual sites 502 of the semiconductor device. In one or more examples, individual reads of the sequencing data can be analyzed to determine a portion of the nucleotide sequence of the reads that corresponds to a spatial identifier. For example, a set of spatial identifier nucleic acid sequences can be analyzed with respect to nucleic acid sequences of the sequencing reads. A portion of the sequencing reads that correspond to a given spatial identifier nucleic acid sequence can be determined. These sequencing reads can then be analyzed with respect to a set of nucleic acid sequences that correspond to compounds that were deposited to the array of sites. In this way, the compound 512 present in an individual site 502 can be identified by determining that sequencing reads corresponding to the individual strands 526 include a sequence that corresponds to both a spatial identifier nucleic acid sequence and a compound identifier nucleic acid sequence.

After pairing the compounds 512 with the electrical data generated in relation to a given site, the process 500 can include analyzing the compound-specific electrical response data 534 produced in response to the electrical signals applied to the sites 502 of the semiconductor device. In one or more examples, the electrical response data 534 can include electrical signals produced in response to performing one or more patch clamp protocols with respect to the array of sites. Additionally, the electrical response data 534 corresponding to an individual site 502 can be analyzed in relation to the compound 512 present in the individual site 502 to determine an impact that the compound 512 has on the biological cells 514 that were present in the individual site 502. To illustrate, the electrical response data 534 can be analyzed to determine an efficacy of the compound 512 with respect to one or more biological conditions. In this way, by depositing hundreds of compounds, thousands of compounds, or more and/or by depositing different concentrations of the compounds within the array of sites, the electrical response data 534 can be analyzed to determine one or more of the compounds that may be candidates for modifying at least one of the functions or structures of the biological cells 514. In at least some examples, the candidate compounds can correspond to potential therapeutic compounds for treating one or more biological conditions that are related to the biological cells 514.

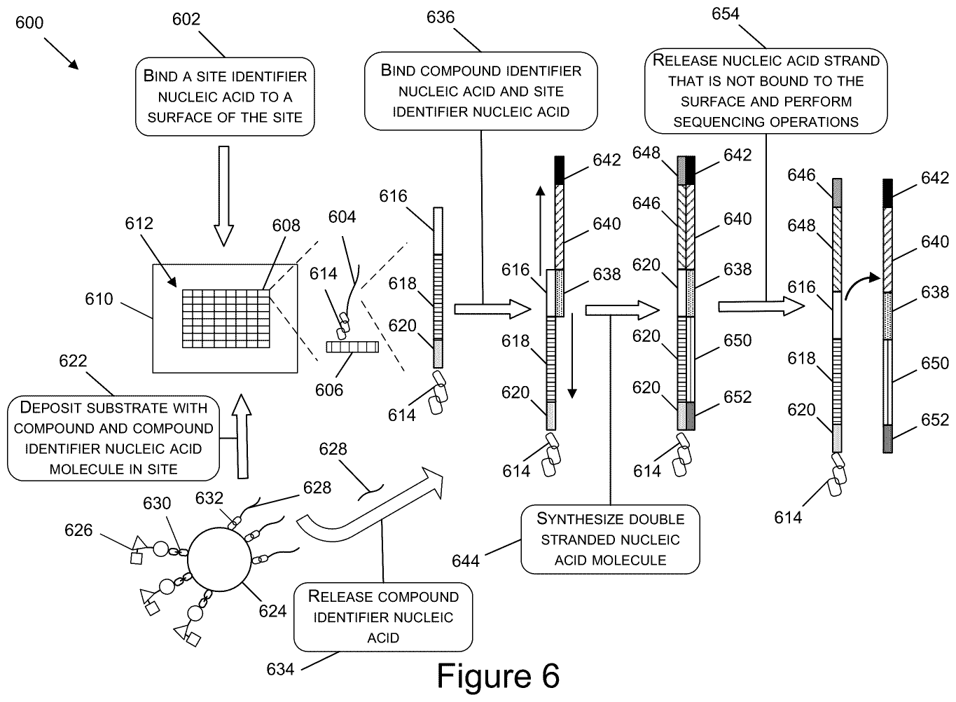

FIG. 6 is a diagram depicting a process 600 to use nucleic acid molecules to track the electrical response of compounds disposed in sites of a semiconductor device, in accordance with one or more example implementations. The process 600 can include, at 602, binding a site identifier nucleic acid 604 to a surface 606 of a site 608 of a semiconductor device 610. In one or more examples, the site 608 can be included in an array of sites 612 that is formed from the semiconductor device 610. In one or more illustrative examples, the site 608, the semiconductor device 610, and the array of sites 612 can correspond to one or more implementations of at least one of the semiconductor device 102, the array of sites 108, and the site 110 described with respect to FIG. 1; the semiconductor device 200, the first site 206, and the second site 208 described with respect to FIG. 2; the semiconductor device 300, the array of sites 304, and the site 306 described with respect to FIG. 3, the semiconductor device 402, the array of sites 404, and the site 410 described with respect to FIG. 4; or the site 502 described with respect to FIG. 5.

The site identifier nucleic acid 604 can be bound to the surface 606 of the site 608 by a first linkage 614. In one or more examples, the first linkage 614 can include one or more molecules having one or more functional groups that are bound to the surface 606 and one or more additional functional groups that are coupled to one or more functional groups of the site identifier nucleic acid 604. In at least some examples, the first linkage 614 can include one or more molecules that can be used to initiate nucleic acid synthesis on the surface 606. In one or more additional examples, the first linkage 614 can include one or more molecular interactions that couple the site identifier nucleic acid 604 to the surface 606. For example, the first linkage 614 can include at least one of electrostatic interactions, hydrogen bond interactions, or van der Waals forces.

The site identifier nucleic acid 604 can have a nucleotide sequence that includes a number of sections. For example, the site identifier nucleic acid 604 can have a nucleotide sequence that includes a first section 616, a second section 618, and a third section 620. The first section 616 can include a nucleotide sequence that corresponds to a hybridization sequence. The hybridization sequence can be included in individual nucleotide sequences of spatial identifier nucleic acids. In at least some examples, the hybridization sequence can be included in the nucleotide sequences of each spatial identifier nucleic acid that is being processed at a given time. In various examples, the hybridization sequence can include a sequence of nucleotides that is complementary to an additional sequence of nucleotides of an additional hybridization sequence of compound identifier nucleic acids. In one or more illustrative examples, the first section 616 can have from 3 nucleotides to 25 nucleotides, from 5 nucleotides to 20 nucleotides, from 5 nucleotides to 15 nucleotides, from 15 nucleotides to 25 nucleotides, from 5 nucleotides to 10 nucleotides, from 10 nucleotides to 15 nucleotides, from 15 nucleotides to 20 nucleotides, or from 20 nucleotides to 25 nucleotides.

The second section 618 can be a site identification sequence and include a sequence of nucleotides that identifies a location of the site 608 within the array of sites 612. In one or more examples, the second section 618 can include a number of subsections. For example, the second section 618 can include 2 subsections, 3 subsections, 4 subsections, 5 subsections, 6 subsections, 7 subsections, 8 subsections, 9 subsections, 10 subsections, or more. The second section 618 can have from 5 nucleotides to 50 nucleotides, from 10 nucleotides to 40 nucleotides, from 10 nucleotides to 20 nucleotides, from 20 nucleotides to 30 nucleotides, from 30 nucleotides to 40 nucleotides, or from 40 nucleotides to 50 nucleotides.

The third section 620 can include a sequence of nucleotides that corresponds to a primer that is used during sequencing operations. The primer can include a sequence of nucleotides by which nucleic acid synthesis can be initiated. The third section 620 can have from 5 nucleotides to 50 nucleotides, from 10 nucleotides to 40 nucleotides, from 10 nucleotides to 20 nucleotides, from 20 nucleotides to 30 nucleotides, from 30 nucleotides to 40 nucleotides, or from 40 nucleotides to 50 nucleotides.

The process 600 can also include, at 622, depositing a compound carrier 624 with a compound 626 and a compound identifier nucleic acid 628 in individual sites 608 of the array of sites 612. In one or more examples, the compound carrier 624 can include a bead. In one or more illustrative examples, the compound carrier 624 can include a bead comprised of at least silica. In one or more additional illustrative examples, the compound carrier 624 can include a bead comprised of one or more magnetic materials. In at least some examples, multiple instances of the compound 626 can be coupled to the compound carrier 624. Further, in various examples, multiple instances of the compound identifier nucleic acid 628 can be coupled to the compound carrier 624.

The compound 626 can include one or more functional groups and can correspond to a molecule that is to undergo testing using electrical signals to determine whether or not the molecule impacts at least one of the functioning or structure of one or more types of biological cells. The compound 626 can be coupled to the compound carrier 624 by a second linkage 630. The second linkage 630 can include one or more molecules that are bound to one or more functional groups of the compound 626 and are bound to a surface of the compound carrier 624. In one or more additional examples, the second linkage 630 can include one or more molecular interactions that couple the compound 626 to the compound carrier 624. For example, the second linkage 630 can include at least one of electrostatic interactions, hydrogen bond interactions, or van der Waals forces.

The compound identifier nucleic acid 628 can include a nucleotide sequence that includes an identifier of the compound 626. The compound identifier nucleic acid 628 can be coupled to the compound carrier 624 using by a third linkage 632. The third linkage 632 can include one or more molecules that are bound to one or more functional groups of the compound identifier nucleic acid 628 and are bound to a surface of the compound carrier 624. In at least some examples, the third linkage 632 can include one or more molecules that can be used to initiate nucleic acid synthesis on the compound carrier 624. In one or more additional examples, the third linkage 632 can include one or more molecular interactions that couple the compound identifier nucleic acid 628 to the compound carrier 624. For example, the third linkage 632 can include at least one of electrostatic interactions, hydrogen bond interactions, or van der Waals forces.

At 634, the process 600 can include releasing the compound identifier nucleic acid 628 from the compound carrier 624. The compound identifier nucleic acid 628 can be released from the compound carrier 624 by cleaving the third linkage 632 coupling the compound carrier 624 and the compound identifier nucleic acid 628. In various examples, the third linkage 632 can be cleaved by at least one of applying one or more wavelengths of electromagnetic radiation to the third linkage 632, by applying one or more enzymes to the third linkage 632, by heating the third linkage 632 above a decomposition temperature for the third linkage 632, or by applying one or more chemical compounds to the third linkage 632. In at least some examples, chemical compounds applied to the third linkage 632 can cause one or more disulfide bonds between the third linkage 632 and the compound identifier nucleic acid 628 to break. In one or more additional examples, chemical compounds applied to the third linkage 632 can cause a pH of an environment in which the third linkage 632 is located to be modified in order to cause the third linkage 632 to be cleaved between the compound carrier 624 and the compound identifier nucleic acid 628.

The process 600 can include, at 636, causing the compound identifier nucleic acid 628 to bind to the site identifier nucleic acid 604. To illustrate, the compound identifier nucleic acid 628 can have a nucleotide sequence that includes a first section 638, a second section 640, and a third section 642. The first section 638 can include a nucleotide sequence that corresponds to a hybridization sequence. The hybridization sequence can be included in individual nucleotide sequences of compound identifier nucleic acids. In at least some examples, the hybridization sequence can be included in the nucleotide sequences of each compound identifier nucleic acid that is being processed at a given time. In various examples, the hybridization sequence can include a sequence of nucleotides that is complementary to the hybridization sequence included in the first section 616 of the site identifier nucleic acid 604. In one or more illustrative examples, the first section 638 can have from 3 nucleotides to 25 nucleotides, from 5 nucleotides to 20 nucleotides, from 5 nucleotides to 15 nucleotides, from 15 nucleotides to 25 nucleotides, from 5 nucleotides to 10 nucleotides, from 10 nucleotides to 15 nucleotides, from 15 nucleotides to 20 nucleotides, or from 20 nucleotides to 25 nucleotides. In one or more examples, the complementary sequences of the first section 616 of the site identifier nucleic acid 604 and the first section 638 of the compound identifier nucleic acid 628 can cause the site identifier nucleic acid 604 to bind to the compound identifier nucleic acid 628.

In addition, the second section 640 of the compound identifier nucleic acid 628 can include a sequence of nucleotides that identifies the compound 626. In one or more examples, the second section 640 can include a number of subsections. For example, the second section 618 can include 2 subsections, 3 subsections, 4 subsections, 5 subsections, 6 subsections, 7 subsections, 8 subsections, 9 subsections, 10 subsections, or more. The second section 640 can have from 5 nucleotides to 50 nucleotides, from 10 nucleotides to 40 nucleotides, from 10 nucleotides to 20 nucleotides, from 20 nucleotides to 30 nucleotides, from 30 nucleotides to 40 nucleotides, or from 40 nucleotides to 50 nucleotides.

Further, the third section 642 of the compound identifier nucleic acid 628 can include a sequence of nucleotides that corresponds to a primer that is used during sequencing operations. The primer can include a sequence of nucleotides by which nucleic acid synthesis can be initiated. The third section 642 can have from 5 nucleotides to 50 nucleotides, from 10 nucleotides to 40 nucleotides, from 10 nucleotides to 20 nucleotides, from 20 nucleotides to 30 nucleotides, from 30 nucleotides to 40 nucleotides, or from 40 nucleotides to 50 nucleotides. In various examples, the primer sequence of the third section 642 of the compound identifier nucleic acid 628 can be different from the primer sequence of the third section 620 of the site identifier nucleic acid 604.

At 644, the process 600 can include synthesizing a double stranded nucleic acid molecule using the site identifier nucleic acid 604 and compound identifier nucleic acid 628 complex. In one or more examples, one or more polymerases in conjunction with dNTP building blocks can be used to extend the nucleotide sequence of the site identifier nucleic acid 604 starting from the first section 616 and adding a fourth section 646 and a fifth section 648 to produce a first strand of the double stranded nucleic acid. The fourth section 646 and the fifth section 648 can comprise an extension of the site identifier nucleic acid 604. The fourth section 646 can include a nucleotide sequence that is complementary to the nucleotide sequence of the second section 640 of the compound identifier nucleic acid 628. In this way, the first strand can include a nucleotide sequence that corresponds to an identifier of the compound 626. Additionally, the fifth section 648 can include a nucleotide sequence that is complementary to the third section 642 of the compound identifier nucleic acid 628.

In one or more additional examples, one or more polymerases in conjunction with dNTP building blocks can be used to extend the nucleotide sequence of the compound identifier nucleic acid 628 starting from the first section 638 and adding a fourth section 650 and a fifth section 652 to produce a second strand of the double stranded nucleic acid. The fourth section 650 and the fifth section 652 can comprise an extension of the compound identifier nucleic acid 628. The fourth section 650 can include a nucleotide sequence that is complementary to the nucleotide sequence of the second section 618 of the site identifier nucleic acid 604. In this way, the second strand can include a nucleotide sequence that corresponds to an identifier of the site 608. Additionally, the fifth section 652 can include a nucleotide sequence that is complementary to the third section 620 of the site identifier nucleic acid 604.

Further, the process 600 can include, at 654, releasing a nucleic acid strand of the double stranded nucleic acid that is not bound to the surface 606 and performing sequencing operations with respect to the released strand. In the illustrative example of FIG. 6, the strand of the double stranded nucleic acid that is not bound to the surface 606 is the second strand that includes the first section 638, the second section 640, the third section 642, the fourth section 650, and the fifth section 652. After sequencing and analyzing sequencing reads produced during the sequencing process that correspond to the second strand, the second strand can be identified as being related to both the site 608 and the compound 626. In this way, electrical signals measured in conjunction with the site 608 can be attributed to the presence of the compound 626 in the site 608 and the impact of the compound 626 on biological cells that were also present in the site 608 when the electrical signals were measured.

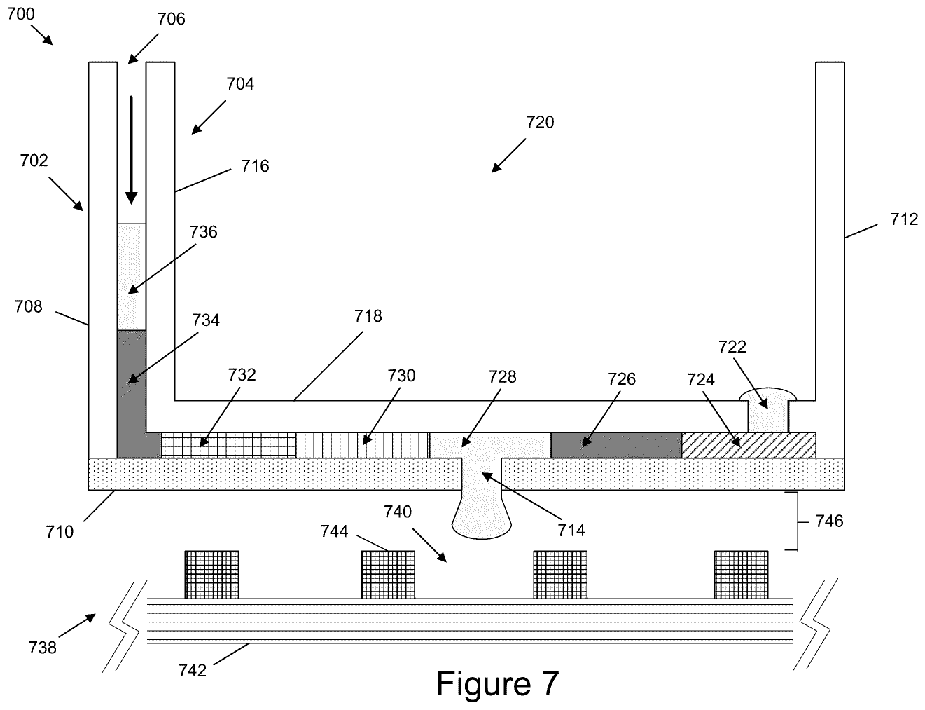

FIG. 7 is a diagram depicting a cross-sectional view of a first example fluid dispensing device 700 to deposit fluids within an array of sites included in a semiconductor device, in accordance with one or more example implementations. The fluid dispensing device 700 can include an outer structure 702 and an inner structure 704. The outer structure 702 and the inner structure 704 can form a channel 706 in which one or more fluids can be disposed. In one or more examples, the outer structure 702 can include one or more sections. For example, the outer structure 702 can include a first outer section 708, a second outer section 710, and a third outer section 712. In various examples, the first outer section 708, the second outer section 710, and the third outer section 712 can be separate sections that are joined together. In one or more illustrative examples, the first outer section 708, the second outer section 710, and the third outer section 712 can be joined by one or more adhesives. In one or more additional illustrative examples, at least a portion of at least two of the first outer section 708, the second outer section 710, or the third outer section 712 can be formed as a continuous piece of material. Further, an opening 714 can be formed in the second outer section 710 to enable fluid to be dispensed from the channel 706.

In one or more examples, the inner structure 704 can be comprised of a first inner section 716 and a second inner section 718. In various examples, the first inner section 716 and the second inner section 718 can be separate sections that are joined together. For example, the first inner section 716 and the second inner section 718 can be joined by one or more adhesives. In one or more additional examples, the first inner section 716 and the second inner section 718 can be a continuous piece of material.

In at least some examples, the first outer section 708, the second outer section 710, the third outer section 712, the first inner section 716, and the second inner section 718 can be comprised of one or more materials. For example, the first outer section 708, the second outer section 710, the third outer section 712, the first inner section 716, and the second inner section 718 can be comprised of one or more polymeric materials. In one or more additional examples, the first outer section 708, the second outer section 710, the third outer section 712, the first inner section 716, and the second inner section 718 can be comprised of one or more silicon-containing materials. In one or more illustrative examples, at least one of the first outer section 708, the second outer section 710, the third outer section 712, the first inner section 716, and the second inner section 718 can be formed from one or more materials that are different from one or more materials used to form the other ones of the first outer section 708, the second outer section 710, the third outer section 712, the first inner section 716, and the second inner section 718. In various examples, the first inner section 716, the second inner section 718, and the third outer section 712 can form an excess fluid area 720. The excess fluid area 720 can capture fluid that is not dispensed from the opening 714.

In various examples, one or more fluids can be dispensed from the fluid dispensing device 700 via the opening 714. In at least some examples, one or more fluids can be dispensed from the opening 714 of the fluid dispensing device 700 as droplets. One or more fluids can be supplied to the fluid dispensing device 700 via the channel 706. Pressure can be applied to the one or more fluids within the channel 706 to cause the one or more fluids to exit the fluid dispensing device 700 via the opening 714. In situations where excess fluid builds up in the channel 706 during the process of dispensing of one or more fluids, at least a portion of the excess fluid can be moved to the excess fluid area 720 through an opening in the second inner section 718.

In one or more examples, the fluids dispensed by the fluid dispensing device 700 can include one or more oils. In one or more additional examples, the fluids dispensed by the fluid dispensing device 700 can include one or more washing or one or more rinsing solutions. In one or more further examples, the fluids dispensed by the fluid dispensing device 700 can include one or more solutions including one or more candidate therapeutic compounds. In still other examples, the fluids dispensed by the fluid dispensing device 700 can include one or more ligand-containing solutions. In one or more illustrative examples, multiple fluids can be dispensed from the fluid dispensing device 700 in accordance with one or more patch clamp protocols. For example, fluids can be supplied to the channel 706 in discrete volumes according to an order specified by the patch clamp protocol. To illustrate, discrete volumes of solutions containing candidate therapeutic compounds can be supplied to the channel in relation to discrete volumes of one or more oils and/or discrete volumes of one or more washing solutions in accordance with a patch clamp protocol. In one or more additional illustrative examples, multiple fluids can be dispensed from the fluid dispensing device 700 to in relation to performing ligand gated ion channel measurements. In these scenarios, discrete volumes of ligand-containing solutions can be supplied to the fluid dispensing device 700 via the channel 706 in relation to washing and/or rinsing solutions, oil solutions, and/or therapeutic-containing solutions in accordance with one or more ligand gated ion channel measurement protocols. In various examples, the ligand-gated ion channel measurements can be produced according to one or more techniques described in Development and Validation of ASIC1a Ligand-Gated Ion Channel Drug Discovery Assays on Automated Patch Clamp Systems, by Marc Rogers, et al.; Biophysics Journal, Volume 120, Issue 3, 338a, published Feb. 12, 2021, which is incorporated by reference herein in its entirety.

In the illustrative example of FIG. 7, the fluid dispensing device 700 can store a first fluid 722, a second fluid 724, a third fluid 726, a fourth fluid 728, a fifth fluid 730, a sixth fluid 732, a seventh fluid 734, and an eighth fluid 736. In one or more examples, at least a portion of the fluids 722, 724, 726, 728, 730, 732, 734, 736 can have a same or similar composition. In one or more additional examples, at least a portion of the fluids 722, 724, 726, 728, 730, 732, 734, 736 can have a different composition with respect to one another. In the illustrative example of FIG. 7, the first fluid 722, the fluid 728, and the eighth fluid 736 can have a same or similar composition and the third fluid 726 and the fluid 734 can have a same or similar composition that is different from the composition of the first fluid 722, the fourth fluid 728, and the eighth fluid 736. Further, the second fluid 724, the fifth fluid 730, and the sixth fluid 732 can have compositions that are different from the composition of the other fluids stored by the fluid dispensing device 700. In one or more illustrative implementations, the first fluid 722, the fourth fluid 728, and the eighth fluid 736 can include a washing solution. Additionally, the second fluid 724 can include a first therapeutic-containing compound and the third fluid 726 can include an oil-containing solution. The fifth fluid 730 can include a second therapeutic-containing compound and the sixth fluid 732 can include a ligand-containing solution. Further, the seventh fluid 734 can include the oil-containing solution.

The fluid dispensing device 700 can dispense fluids onto or into a fluid accepting device 738. The fluid accepting device 738 can include one or more sites 740 for holding discrete volumes of liquid. In one or more examples, the sites 740 can be formed by one or more substrates 742 and a number of walls 744. In one or more illustrative examples, the fluid accepting device 738 can include a well plate and the individual sites 740 can correspond to individual wells of the well plate. Additionally, the fluid accepting device 738 can include one or more semiconductor devices. For example, the fluid accepting device 738 can include one or more implementations of the semiconductor devices described in U.S. patent application Ser. No. 18/769,215 filed Jul. 10, 2024, which is incorporated by reference herein in its entirety. In various examples, a gap 746 can be disposed between the fluid dispensing device 700 and a fluid accepting device 738. In situations where patch clamp measurements are being performed by the fluid accepting device 738, the gap 746 can be filled with oil when the sites are filled with candidate therapeutic compound-containing solutions during the patch clamp operations.

Although the illustrative example of FIG. 7 shows the fluid dispensing device 700 having a single opening 714 for dispensing fluids, in one or more additional implementations, the fluid dispensing device 700 can include multiple openings for dispensing fluid. In these scenarios, multiple fluids can be dispensed from the fluid dispensing device 700 contemporaneously. In other examples, a same fluid can be dispensed to multiple sites of a fluid accepting device 738 in situations where the fluid dispensing device 700 includes multiple openings for dispensing fluids. Further, although the illustrative example of FIG. 7 shows that fluid is being dispensed through one or more openings in the fluid dispensing device 700, in one or more additional examples, the fluid dispensing device 700 can include one or more pipettes or other fluid delivery devices that are coupled to the channel 706 in order to dispense fluid from the fluid dispensing device 700. In one or more further examples, the fluid dispensing device 700 can include one or more valves that are coupled to the channel 706 via the opening 714 to dispense fluid from the fluid dispensing device 700. The one or more valves can be actuated by at least one of magnetic actuation, electrostatic actuation, thermal expansion, or pneumatic techniques. In still other examples, the one or more valves can include hydrogels or other phase change materials. In various examples, the fluid dispensing device 700 can be manufactured according to silicon-based micro-electromechanical systems (MEMS) processes, such as those described in Ning Gao, XiuJun (James) Li, 5—Controlled drug delivery using microdevices, Editor(s): Xiujun (James) Li, Yu Zhou, In Woodhead Publishing Series in Biomaterials, Microfluidic Devices for Biomedical Applications (Second Edition), Woodhead Publishing, 2021, Pages 205-223, ISBN 9780128199718, which is incorporated by reference herein in its entirety.

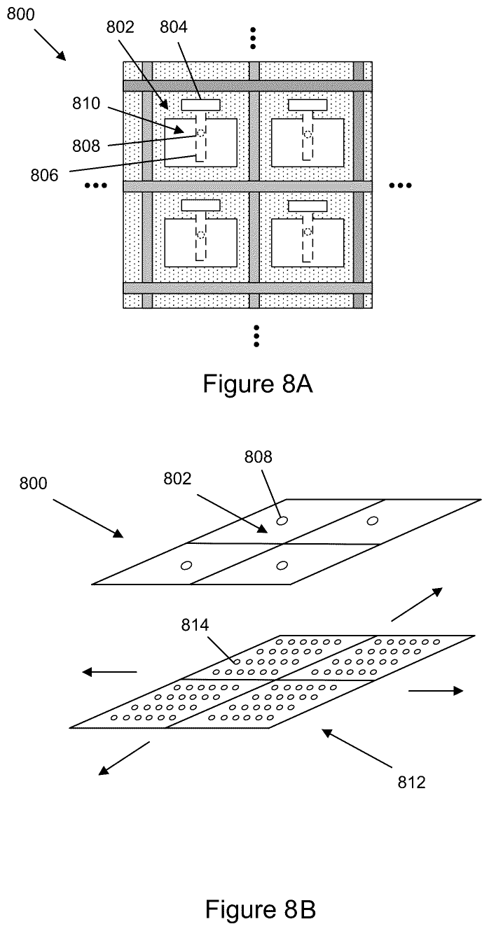

FIG. 8A is a diagram depicting a top view of an example fluid dispensing device 800, in accordance with one or more example implementations. The example fluid dispensing device 800 can include a number of sections 802. The individual sections 802 can include an inlet 804 of a channel 806. An opening 808 can be formed in the channel 806 to dispense fluids that are supplied to the channel 806. Additionally, the individual sections 802 can include an excess fluid area 810. The channel 806 and the excess fluid area 810 can be formed by one or more outer structures or one or more inner structures. Although the illustrative example of FIG. 8A shows four sections of the fluid dispensing device 800, in various implementations, the fluid dispensing device 800 can include fewer than four sections or more than four sections.

In one or more examples, the individual sections 802 can have a surface area from about 100 millimeters squared (mm²) to about 50 centimeters squared (cm²), from about 500 mm² to about 25 cm², from about 1 cm² to about 10 cm², from about 100 mm² to about 1 cm², from about 500 mm² to about 25 cm², from about 25 cm² to about 50 cm², from about 1 cm² to about 10 cm², from about 10 cm² to about 20 cm², from about 20 cm² to about 30 cm², from about 30 cm² to about 40 cm², or from about 40 cm² to about 50 cm². In one or more additional examples, the opening 808 can have a diameter from about 0.5 μm to about 25 μm, from about 1 μm to about 10 μm, from about 10 μm to about 20 μm, from about 1 μm to about 5 μm, from about 5 μm to about 10 μm, from about 10 μm to about 15 μm, from about 15 μm to about 20 μm, or from about 20 μm to about 25 μm.

In one or more illustrative examples, the example fluid dispensing device 800 can include the fluid dispensing device 700. In these scenarios, individual sections of the fluid dispensing device 800 can include the components of the fluid dispensing device 700. For example, individual sections of the fluid dispensing device 800 can include an outer structure 702 including a first outer section 708, a second outer section 710, and a third outer section 712 and an inner structure 704 including a first inner section 716 and a second inner section 718 with the outer structure 702 and the inner structure 704 forming a channel 706 and the opening 714 corresponding to the opening 808.

FIG. 8B is a view of the fluid dispensing device 800 and a fluid accepting device 812. The fluid accepting device 812 can include a number of sites 814. In at least some examples, the fluid accepting device 812 can correspond to one or more implementations of the fluid accepting device 738 described with respect to FIG. 7. In one or more examples, the fluid accepting device 812 can be moved in a number of directions to align the sites 814 of the fluid accepting device 812 with the openings 808 of the fluid dispensing device 800. In one or more illustrative examples, openings 808 of the fluid dispensing device 800 can be aligned with a first set of sites 814 of the fluid accepting device 812. Fluid can be dispensed from the openings 808 into the first set of sites 814. After a volume of fluid is dispensed from the openings 808 into the first set of sites 814, the fluid accepting device 812 can be moved such that a different set of sites 814 is aligned with the openings 808. A volume of the same fluid that was dispensed into the first set of sites or a volume of a different fluid that was dispensed into the first set of sites can then be dispensed into the second set of sites 814.

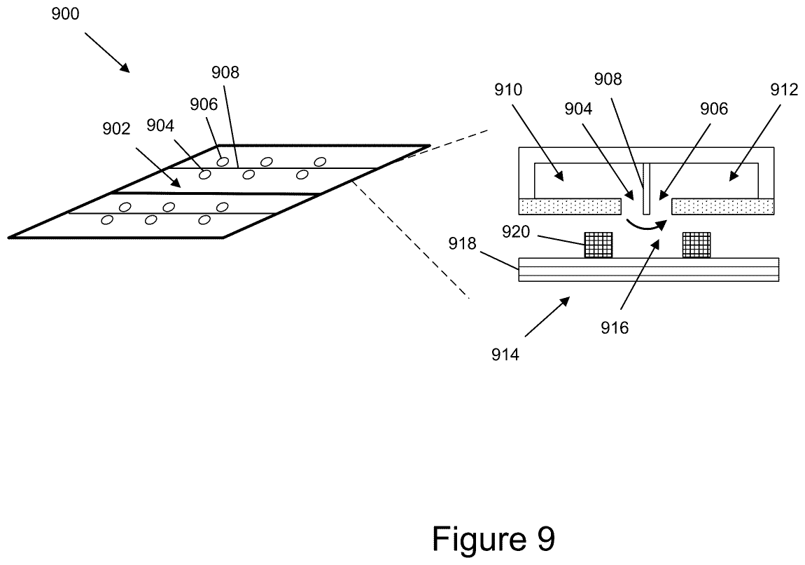

FIG. 9 is a diagram depicting a top view and a cross-sectional view of a second example fluid dispensing device 900 to deposit fluids within an array of sites included in a semiconductor device, in accordance with one or more example implementations. The fluid dispensing device 900 can include a number of sections 902. The individual sections 902 can be constructed similar to the sections 802 described in relation to FIG. 8, but with the sections 902 including multiple openings rather than a single opening. In the illustrative example of FIG. 9, the sections 902 include at least a first opening 904 and a second opening 906 that are separated by a dividing structure 908. The first opening 904 is used to dispense fluid from the fluid dispensing device 900 and the second opening 906 is used to bring fluid into the fluid dispensing device 900.

In one or more examples, the fluid dispensing device 900 can include a first channel 910 that includes the first opening 904 and a second channel 912 that includes the second opening 906. In various examples, positive pressure can be applied to fluid disposed in the first channel 910 to cause the fluid to be dispensed from the fluid dispensing device 900 via the first opening 904. Additionally, negative pressure can be applied to the second channel 912 to cause fluid to be drawn into the second channel 912 via the second opening 906. In one or more illustrative examples, fluid in the first channel 910 can be dispensed onto a fluid accepting device 914 that includes a number of sites 916 formed by one or more substrates 918 and a number of walls 920. In at least some examples, after one or more fluids are dispensed onto the fluid accepting device 914 via the first opening 904, at least a portion of the fluid within the sites 916 can be drawn into the second channel 912 via the second opening 906. In one or more additional illustrative examples, the fluid dispensing device 900 can be used to perform one or more patch clamp protocols. In addition to dispensing solutions that include candidate therapeutic compounds onto the fluid accepting device 914, the fluid dispensing device 900 can also perform one or more washing operations as part of the patch clamp protocols. During the washing operations, the fluid dispensing device 900 can dispense one or more fluids including one or more washing solutions onto the fluid accepting device 914 via the first opening 904 and draw at least a portion of the one or more washing solutions into the second channel 912 via the second opening 906. In one or more further illustrative examples, the one or more washing solutions can be drawn into the second channel 912 after having been dispensed onto the fluid accepting device 914 for a period of time. In still other examples, the dispensing of the one or more washing solutions via the first opening 904 and the drawing of the one or more washing solutions into the second channel 912 via the second opening 906 can be a continuous process, a semi-continuous process, or a substantially continuous process.

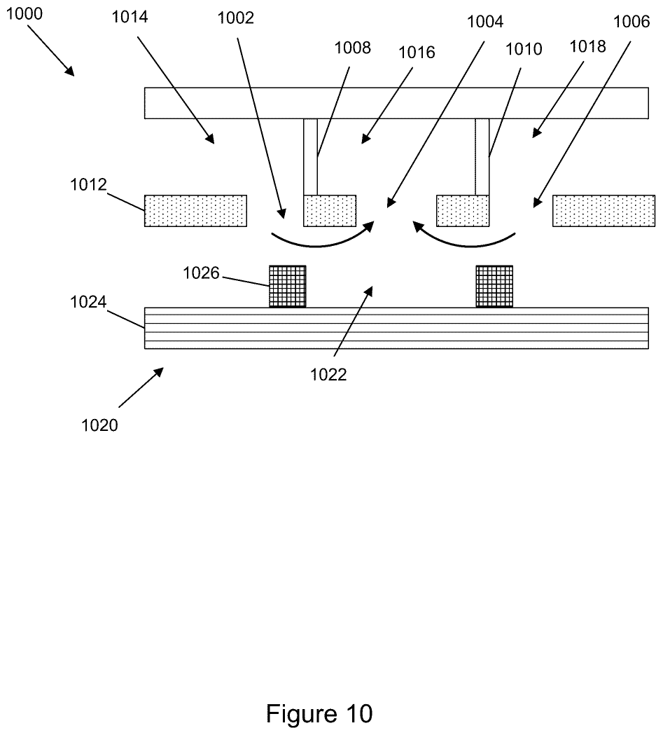

FIG. 10 is a diagram depicting a cross-sectional view of an additional example outlet arrangement 1000 for a device that dispenses fluid to an array of sites in a semiconductor device, in accordance with one or more example implementations. The additional example outlet arrangement 1000 can be implemented with respect to one or more implementations of the fluid dispensing device 700 described in relation to FIG. 7, the fluid dispensing device 800 described in relation to FIG. 8, and the fluid dispensing device 900 described in relation to FIG. 9.

The additional example outlet arrangement 1000 can include at least a first opening 1002, a second opening 1004, and a third opening 1006. The first opening 1002 and the second opening 1004 can be separated by a first dividing structure 1008. The second opening 1004 and the third opening 1006 can be separated by a second dividing structure 1010. The first opening 1002, the second opening 1004, and the third opening 1006 can be formed by an outer structure 1012 of a fluid dispensing device.

The first opening 1002 can be used to dispense one or more first fluids from the fluid dispensing device and the third opening 1006 can be used to dispense one or more second fluids from the fluid dispensing device. In one or more examples, the one or more first fluids and the one or more second fluids can be dispensing contemporaneously. In one or more additional examples, the one or more first fluids and the one or more second fluids can be dispensed at separate times. The second opening 1004 can be used to draw fluid into the fluid dispensing device.

In one or more examples, the additional example outlet arrangement 1000 can include a first channel 1014 that includes the first opening 1002, a second channel 1016 that includes the second opening 1004, and a third channel 1018 that includes the third opening 1006. In various examples, positive pressure can be applied to a first fluid disposed in the first channel 1014 to cause the first fluid to be dispensed from the fluid dispensing device via the first opening 1002.

Additionally, positive pressure can be applied to a second liquid disposed in the third channel 1018 to cause the second fluid to be dispensed from the fluid dispensing device via the third opening 1006. Further, negative pressure can be applied to the second channel 1016 to cause at least a portion of the first fluid or at least a portion of the second fluid to be drawn into the second channel 1016 via the second opening 1004.

In one or more illustrative examples, first fluid in the first channel 1014 and second fluid in the third channel 1018 can be dispensed onto a fluid accepting device 1020 that includes a number of sites 1022 formed by one or more substrates 1024 and a number of walls 1026. In at least some examples, after one or more fluids are dispensed onto the fluid accepting device 1020 via the first opening 1002 and/or the third opening 1006, at least a portion of the fluid within the sites 1022 can be drawn into the second channel 1016 via the second opening 1004. In one or more additional illustrative examples, the additional example outlet arrangement 1000 of a fluid dispensing device can be used to perform one or more patch clamp protocols. In addition to dispensing solutions that include candidate therapeutic compounds onto the fluid accepting device 1020, the fluid dispensing device can also use the additional example outlet arrangement 1000 to perform one or more washing operations as part of the patch clamp protocols. During the washing operations, the fluid dispensing device can use the additional example outlet arrangement 1000 to dispense one or more fluids including one or more washing solutions onto the fluid accepting device 1020 via the first opening 1002 or the third opening 1006 and draw at least a portion of the one or more washing solutions into the second channel 1016 via the second opening 1004. In one or more further illustrative examples, the one or more washing solutions can be drawn into the second channel 1016 after having been dispensed onto the fluid accepting device 1020 for a period of time. In still other examples, the dispensing of the one or more washing solutions via the first opening 1002 or the third opening 1006 and the drawing of the one or more washing solutions into the second channel 1016 via the second opening 1004 can be a continuous process, a semi-continuous process, or a substantially continuous process.

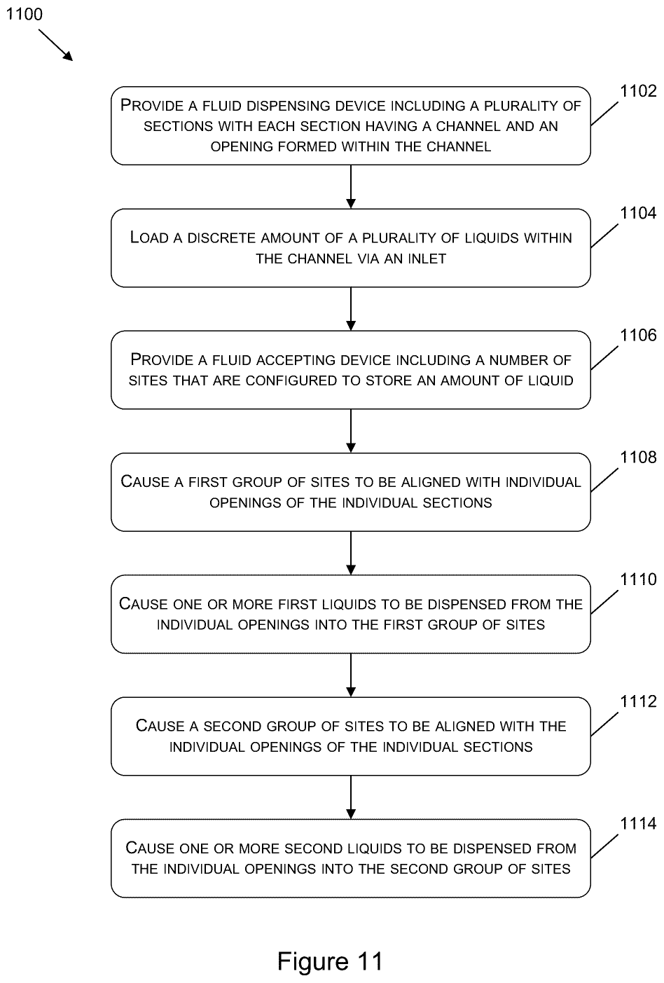

FIG. 11 is a flow diagram of a process 1100 to dispense fluids from a fluid accepting device to a fluid accepting device, in accordance with one or more example implementations. At 1102, the process 1100 can include providing a fluid dispensing device including a plurality of sections with individual sections of the plurality of sections including an outer structure and an inner structure. The outer structure and the inner structure can form a channel and an inlet for the channel. The inlet can be disposed at least substantially perpendicular with respect to the channel. The channel can include an opening to dispense liquid.

In addition, the process 1100 can include, at 1104, loading a discrete amount of a plurality of liquids within the channel of the individual sections via the inlet of the individual sections. The plurality of solutions can include an aqueous solution comprising one or more compounds, a washing solution, and an oil-containing solution. In one or more examples, the one or more first liquids can include a first candidate therapeutic compound. In addition, the one or more second liquids can include a second candidate therapeutic compound. Further, at least one of the one or more first liquids or the one or more second liquids can include a ligand that modifies one or more ion channels of a biological cell.

Further, the process 1100 can include, at 1106, providing a fluid accepting device including a number of sites. Individual sites of the number of sites can be configured to store an amount of at least one liquid of the plurality of liquids. In one or more examples, the fluid accepting device can include a well plate comprising at least 24 individual wells. In one or more additional examples, the fluid accepting device includes a semiconductor device having a plurality of sites, with individual sites can include a well formed by a plurality of walls and a substrate of the semiconductor device. The fluid accepting device can be disposed at least substantially parallel with respect to the fluid dispensing device.

The process 1100, at 1108, can also include causing a first group of sites of the number of sites to be aligned with individual openings of the individual sections. Additionally, at 1110, the process 1100 can include causing one or more first liquids of the plurality of liquids to be dispensed from the individual openings into the first group of sites. At 1112, the process 1100 can include causing a second group of sites of the number of sites to be aligned with the individual openings of the individual sections. The process 1100 can include, at 1114, causing one or more second liquids of the plurality of liquids to be dispensed from the individual openings into the second group of sites.

In various examples, the dispensing of liquid from the fluid dispensing device and the movement of the fluid accepting device can be part of a number of cycles with each cycle including at least one fluid dispensing operation and at least one fluid accepting device movement operation. Individual cycles can include dispensing a buffer solution comprising at least one candidate therapeutic compound into one or more sites of the number of sites and after dispensing the buffer solution; dispensing one or more rinsing solutions into the one or more sites. In at least some examples, the buffer solution can include one or more biological cells. In one or more examples, the individual cycles can include dispensing an oil-containing solution into the one or more sites after dispensing the buffer solution and before dispensing the one or more rinsing solutions into the one or more sites. In one or more further examples, the individual cycles can include applying at least one of voltage signals or current signals to the number of sites after dispensing the buffer solution and before dispensing the one or more rinsing solutions. Applying electrical signals to fluid disposed in the fluid accepting device can be part of one or more patch clamp operations. In still other examples, individual cycles can include moving the fluid accepting device from about 100 μm to about 400 μm in a lateral direction.

A numbered non-limiting list of aspects of the present subject matter is presented below.

Aspect 1 is a method comprising: providing a semiconductor device including a substrate having one or more layers and an array of sites formed on the substrate, wherein individual sites include, a number of walls and the one or more layers of the substrate include a complementary metal oxide semiconductor (CMOS) layer; forming one or more first nucleic acid molecules within individual sites of the array of sites, wherein individual nucleotide sequences of the one or more first nucleic acid molecules include an identifier of a location of an individual site within the array of sites; dispensing a solution on the semiconductor device, the solution including a plurality of compound carriers with one or more second nucleic acid molecules coupled to an individual compound carrier and one or more test compounds being coupled to the individual compound carrier, the one or more second nucleic acid molecules including nucleotide sequences that include an identifier of the one or more test compounds; providing a number of biological cells to the semiconductor device; applying at least one of voltage or current to one or more electrodes included in the individual sites of the array of sites while the one or more test compounds and one or more biological cells of the number of biological cells are disposed in the individual sites; and measuring electrical signals produced in response to the at least one of voltage or current being applied to the one or more electrodes.

In Aspect 2, the subject matter of Aspect 1 includes, wherein the one or more first nucleic acid molecules include: a site identification sequence that indicates the location of the individual site within the array of sites; a primer sequence; and a hybridization sequence; wherein the site identification sequence is located between the primer sequence and the hybridization sequence.

In Aspect 3, the subject matter of any one of Aspects 1-2 includes, wherein the individual site includes a base surface formed from a plurality of materials and the plurality of materials include one or more hydrophilic materials.

In Aspect 4, the subject matter of Aspect 3 includes, wherein the base surface of the individual site includes a first section formed from one or more first materials, a second section formed from one or more second materials, and a third section formed from one or more third materials.

In Aspect 5, the subject matter of Aspect 4 includes, wherein: the one or more first materials are configured to attract the individual compound carrier toward the first section of the base surface; the one or more second materials are configured to bind the one or more first nucleic acid molecules to the second section of the base surface; and the one or more third materials are configured to attract the number of biological cells to the third section of the base surface.

In Aspect 6, the subject matter of any one of Aspects 3-5 includes, wherein the one or more first nucleic acid molecules are bound to the base surface according to a first binding interaction and the one or more second nucleic acid molecules are bound to the base surface according to a second binding interaction.

In Aspect 7, the subject matter of Aspect 6 includes, wherein: the first binding interaction is different from the second binding interaction; and the first binding interaction and the second binding interaction include at least two of a photocleavable binding interaction, a heat cleavable binding interaction, an enzymatically cleavable binding interaction, an acid cleavable binding interaction, or one or more disulfide bonds.

In Aspect 8, the subject matter of any one of Aspects 1-7 includes, causing the one or more second nucleic acid molecules to be released from the individual compound carrier; and causing the one or more biological cells to be released from the individual compound carrier.

In Aspect 9, the subject matter of Aspect 8 includes, applying one or more linkage cleaving techniques to cause the one or more second nucleic acid molecules and the one or more biological cells are released from the individual compound carrier.

In Aspect 10, the subject matter of Aspect 9 includes, causing a first hybridization sequence of a first nucleic acid molecule of the one or more first nucleic acid molecules to bind to a second hybridization sequence of a second nucleic acid molecule of the one or more second nucleic acid molecules; wherein the first hybridization sequence includes a first nucleotide sequence and the second hybridization sequence includes a second nucleotide sequence that is complementary with respect to the first nucleotide sequence.

In Aspect 11, the subject matter of Aspect 10 includes, providing one or more solutions to the individual site, wherein the solution includes nucleic acid polymerase enzyme and deoxynucleotide triphosphates; synthesizing a first extension of the first nucleic acid molecule to produce a first strand of a double stranded nucleic acid; and synthesizing a second extension of the second nucleic acid molecule to produce a second strand of the double stranded nucleic acid.

In Aspect 12, the subject matter of Aspect 11 includes, wherein the first strand includes: a first primer sequence; a first identifier sequence t that includes a nucleotide sequence that is complementary to an additional nucleotide sequence that includes an identifier of the location of the individual site within the array of sites; the first hybridization sequence; a second identifier sequence that includes a nucleotide sequence that includes an identifier of a test compound of the one or more test compounds; and a second primer segment.

In Aspect 13, the subject matter of Aspect 12 includes, wherein the second strand includes: an additional first primer sequence that includes a nucleotide sequence that is complementary to the first primer sequence; a third identifier sequence that includes the additional nucleotide sequence that includes the identifier of the location of the individual site; the second hybridization sequence; a fourth identifier sequence that includes a further nucleotide sequence that is complementary to the nucleotide sequence that includes the identifier of the test compound; and an additional second primer sequence that includes a nucleotide sequence that is complementary to the second primer sequence t.

In Aspect 14, the subject matter of any one of Aspects 11-13 includes, applying one or more denaturation processes to cause the first strand to separate from the second strand; collecting the second strand; and performing one or more sequencing operations with respect to the second strand to produce sequencing data corresponding to the second strand.

In Aspect 15, the subject matter of Aspect 14 includes, analyzing sequencing reads included in the sequencing data to determine that a test compound of the one or more test compounds was present in the individual site; and collecting electrical response data for the individual site that indicates the electrical signals produced in response to the at least one of voltage or current being applied to the one or more electrodes.

In Aspect 16, the subject matter of Aspect 15 includes, analyzing the electrical response data to determine an efficacy of the test compound with respect to one or more biological conditions related to the number of biological cells.

In Aspect 17, the subject matter of any one of Aspects 1-16 includes, performing an enzymatic nucleic acid synthesis process or a light-based nucleic acid synthesis process to produce the one or more first nucleic acid molecules within the individual site; wherein the one or more first nucleic acid molecules are synthesized using one or more initiator molecules that are bound to a base surface of the individual sites.

In Aspect 18, the subject matter of any one of Aspects 1-17 includes, after dispensing the solution on the semiconductor device that includes the plurality of compound carriers with the one or more second nucleic acid molecules coupled to the individual compound carrier and the one or more test compounds being coupled to the individual compound carrier, dispensing an oil solution on the semiconductor device; wherein the oil solution is dispensed on the semiconductor device before applying the at least one of the voltage or the current to the one or more electrodes included in the individual site.

In Aspect 19, the subject matter of any one of Aspects 1-18 includes, wherein the compound carrier includes a bead and a diameter of at least a portion of the individual site is no greater than 1.5 times a diameter of the bead.

In Aspect 20, the subject matter of any one of Aspects 1-19 includes, wherein the CMOS layer includes circuitry to (i) apply at least one of the voltage or the current to the one or more electrodes included in the individual sites and (ii) measure the electrical signals produced in response to the at least one of voltage or current being applied to the one or more electrodes.

Aspect 21 is an article comprising: a bead; one or more instances of a compound bound to the bead, wherein the compound has an average molecular weight that is no greater than 1000 grams/mol; and one or more nucleic acids bound to the bead, the one or more nucleic acids including a segment having a nucleotide sequence that corresponds to an identifier of the compound.

In Aspect 22, the subject matter of Aspect 21 includes, wherein the bead comprises silica or a magnetic material.

In Aspect 23, the subject matter of any one of Aspects 21-22 includes, wherein the compound is bound to the bead by a first linkage and the one or more nucleic acids are bound to the bead by a second linkage.

In Aspect 24, the subject matter of Aspect 23 includes, wherein the first linkage and the second linkage can be cleaved by a light-based process, an enzymatic process, a temperature based process, or a chemical based process.

In Aspect 25, the subject matter of any one of Aspects 21-24 includes, wherein the one or more nucleic acids include a first additional segment that includes a first additional nucleotide sequence that corresponds to a hybridization sequence and a second additional nucleotide sequence that corresponds to a primer sequence.

In Aspect 26, the subject matter of any one of Aspects 24-25 includes, wherein the first linkage and the second linkage include one or more linker molecules.

In Aspect 27, the subject matter of any one of Aspects 21-26 includes, wherein the bead has a diameter from about 50 micrometers to about 800 micrometers.

Aspect 28 is a device comprising: an outer structure including a first section, a second section that is joined to the first section and disposed at least substantially perpendicular with respect to the first section, and a third section that is joined to the second section and is disposed at least substantially parallel with respect to the first section; an inner structure including a first additional section that is disposed at least substantially parallel with respect to the first section and a second additional section that is disposed at least substantially perpendicular with respect to the first additional section; wherein: the first section and the first additional section form a first portion of a channel and the second section and the second additional section form a second portion of the channel that is disposed at least substantially perpendicular with respect to the first portion of the channel; an opening is disposed in the second section and is in fluid communication with the second portion of the channel; and an excess fluid area is disposed above the channel and is formed by the first additional section and the second additional section of the inner structure and the third section of the outer structure.

In Aspect 29, the subject matter of Aspect 28 includes, wherein the outer structure and the inner structure are comprised of one or more polymeric materials.

In Aspect 30, the subject matter of any one of Aspects 28-29 includes, wherein a diameter of the opening is no greater than 250 micrometers.

In Aspect 31, the subject matter of any one of Aspects 28-30 includes, wherein: an additional opening is disposed in the second section of the outer structure; positive pressure is applied to dispense fluid located in the channel out of the opening; and negative pressure is applied to draw additional fluid into the channel via the additional opening.

In Aspect 32, the subject matter of any one of Aspects 28-31 includes, a plurality of sections with individual sections of the plurality of sections including an individual outer structure, an individual inner structure, at least one opening, and an individual excess fluid area.

In Aspect 33, the subject matter of any one of Aspects 28-32 includes, wherein the opening is coupled to a valve and the valve is actuated to dispense fluid from the channel.

Aspect 34 is a method comprising: providing a fluid dispensing device including a plurality of sections with individual sections of the plurality of sections including an outer structure and an inner structure, the outer structure and the inner structure forming a channel and an inlet for the channel, the inlet being disposed at least substantially perpendicular with respect to the channel, and the channel including an opening to dispense liquid; loading a discrete amount of a plurality of liquids within the channel of the individual sections via the inlet of the individual sections; providing a fluid accepting device including a number of sites, individual sites of the number of sites being configured to store an amount of at least one liquid of the plurality of liquids; causing a first group of sites of the number of sites to be aligned with individual openings of the individual sections; causing one or more first liquids of the plurality of liquids to be dispensed from the individual openings into the first group of sites; causing a second group of sites of the number of sites to be aligned with the individual openings of the individual sections; and causing one or more second liquids of the plurality of liquids to be dispensed from the individual openings into the second group of sites.

In Aspect 35, the subject matter of Aspect 34 includes, wherein the plurality of liquids include an aqueous solution comprising one or more compounds, a washing solution, and an oil-containing solution.

In Aspect 36, the subject matter of any one of Aspects 34-35 includes, wherein the one or more first liquids include a first candidate therapeutic compound and the one or more second liquids include a second candidate therapeutic compound.

In Aspect 37, the subject matter of any one of Aspects 34-36 includes, wherein at least one of the one or more first liquids or the one or more second liquids include a ligand that modifies one or more ion channels of a biological cell.

In Aspect 38, the subject matter of any one of Aspects 34-37 includes, performing a number of cycles of dispensing liquid from the fluid dispensing device and movement of the fluid accepting device after dispensing the liquid from the fluid dispensing device, wherein individual cycles of the number of cycles include: dispensing a buffer solution comprising at least one candidate therapeutic compound into one or more sites of the number of sites; and after dispensing the buffer solution; dispensing one or more rinsing solutions into the one or more sites.

In Aspect 39, the subject matter of Aspect 38 includes, wherein the individual cycles of the number of cycles include dispensing an oil-containing solution into the one or more sites after dispensing the buffer solution and before dispensing the one or more rinsing solutions into the one or more sites.

In Aspect 40, the subject matter of any one of Aspects 38-39 includes, wherein the buffer solution includes one or more biological cells.

In Aspect 41, the subject matter of any one of Aspects 38-40 includes, wherein the individual cycles of the number of cycles include applying at least one of voltage signals or current signals to the number of sites after dispensing the buffer solution and before dispensing the one or more rinsing solutions.

In Aspect 42, the subject matter of any one of Aspects 38-41 includes, wherein the individual cycles of the number of cycles include moving the fluid accepting device from about 100 μm to about 400 μm in a lateral direction.

In Aspect 43, the subject matter of any one of Aspects 34-42 includes, wherein the fluid accepting device includes a well plate comprising at least 24 individual wells.

In Aspect 44, the subject matter of any one of Aspects 34-43 includes, wherein the fluid accepting device includes a semiconductor device having a plurality of sites, with individual sites including a well formed by a plurality of walls and a substrate of the semiconductor device.

Aspect 45 is a semiconductor device comprising: a substrate having one or more layers and an array of sites formed on the substrate, wherein the one or more layers of the substrate include a complementary metal oxide semiconductor (CMOS) layer and individual sites include: a number of walls comprised of one or more hydrophobic materials; and a base surface comprised of one or more hydrophilic materials, the base surface including a first portion having one or more first materials to couple one or more nucleic acids to the first section and a second portion having one or more second materials to couple a bead to the second section.

In Aspect 46, the subject matter of Aspect 45 includes, wherein the one or

In Aspect 47, the subject matter of Aspect 46, wherein the one or more photoresist materials include at least one of an SU-8 photoresist, a polytetrafluoroethylene (PTFE), one or more polyimides, one or more epoxy-based polymers, benzocyclobutene (BCB), polydimethylsiloxane (PDMS), polymethylmethacrylate (PMMA), or octadecyltrimethoxysilane.

In Aspect 48, the subject matter of any one of Aspects 45-47, wherein the base surface of the individual sites includes a third section comprised of one or more third materials to couple one or more biological cells to the third section.

In Aspect 49, the subject matter of any one of Aspects 45-48, wherein the individual sites are formed according to a pattern including a first section in fluid communication with a second section.

In Aspect 50, the subject matter of Aspect 49, wherein the first section has at least a substantially circular shape.

In Aspect 51, the subject matter of Aspect 50, wherein the first section has a diameter that is no greater than about 1.5 times a diameter of the bead.

In Aspect 52, the subject matter of any one of Aspects 50-51, wherein the second section has at least a substantially circular shape and has an additional diameter that is less than the diameter of the first section.

The above detailed description includes references to the accompanying drawings, which form a part of the detailed description. The drawings show, by way of illustration, specific embodiments in which the invention may be practiced. These implementations are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other implementations can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed implementation. Thus, the following claims are hereby incorporated into the Detailed Description as examples or implementations, with each claim standing on its own as a separate implementation, and it is contemplated that such implementations can be combined with each other in various combinations or permutations. The scope of the invention should be determined with reference to the appended claims, along with the full scope of equivalents to which such claims are entitled.

What is claimed is:

1. A method comprising:
providing a semiconductor device including a substrate having one or more layers and an array of sites formed on the substrate, wherein individual sites include a number of walls and the one or more layers of the substrate include a complementary metal oxide semiconductor (CMOS) layer;
forming one or more first nucleic acid molecules within individual sites of the array of sites, wherein individual nucleotide sequences of the one or more first nucleic acid molecules include an identifier of a location of an individual site within the array of sites;
dispensing a solution on the semiconductor device, the solution including a plurality of compound carriers with one or more second nucleic acid molecules coupled to an individual compound carrier and one or more test compounds being coupled to the individual compound carrier, the one or more second nucleic acid molecules including nucleotide sequences that include an identifier of the one or more test compounds;
providing a number of biological cells to the semiconductor device;
applying at least one of voltage or current to one or more electrodes included in the individual sites of the array of sites while the one or more test compounds and one or more biological cells of the number of biological cells are disposed in the individual sites; and
measuring electrical signals produced in response to the at least one of voltage or current being applied to the one or more electrodes.

2. The method of claim 1, wherein the one or more first nucleic acid molecules include:
a site identification sequence that indicates the location of the individual site within the array of sites;
a primer sequence; and
a hybridization sequence;
wherein the site identification sequence is located between the primer sequence and the hybridization sequence.

3. The method of claim 1, wherein the individual site includes a base surface formed from a plurality of materials and the plurality of materials include one or more hydrophilic materials.

4. The method of claim 3, wherein the base surface of the individual site includes a first portion formed from one or more first materials, a second portion formed from one or more second materials, and a third portion formed from one or more third materials.

5. The method of claim 4, wherein:
the one or more first materials are configured to attract the individual compound carrier toward the first section of the base surface;
the one or more second materials are configured to bind the one or more first nucleic acid molecules to the second section of the base surface; and
the one or more third materials are configured to attract the number of biological cells to the third section of the base surface.

6. The method of claim 3, wherein the one or more first nucleic acid molecules are bound to the base surface according to a first binding interaction and the one or more second nucleic acid molecules are bound to the base surface according to a second binding interaction.

7. The method of claim 6, wherein:
the first binding interaction is different from the second binding interaction; and
the first binding interaction and the second binding interaction include at least two of a photocleavable binding interaction, a heat cleavable binding interaction, an enzymatically cleavable binding interaction, an acid cleavable binding interaction, or one or more disulfide bonds.

8. The method of claim 1, comprising:
causing the one or more second nucleic acid molecules to be released from the individual compound carrier; and
causing the one or more biological cells to be released from the individual compound carrier.

9. The method of claim 8, comprising:
applying one or more linkage cleaving techniques to cause the one or more second nucleic acid molecules and the one or more biological cells to be released from the individual compound carrier.

10. The method of claim 9, comprising:
causing a first hybridization sequence of a first nucleic acid molecule of the one or more first nucleic acid molecules to bind to a second hybridization sequence of a second nucleic acid molecule of the one or more second nucleic acid molecules;
wherein the first hybridization sequence includes a first nucleotide sequence and the second hybridization sequence includes a second nucleotide sequence that is complementary with respect to the first nucleotide sequence.

11. The method of claim 10, comprising:
providing one or more solutions to the individual site, wherein the solution includes nucleic acid polymerase enzyme and deoxynucleotide triphosphates;
synthesizing a first extension of the first nucleic acid molecule to produce a first strand of a double stranded nucleic acid; and
synthesizing a second extension of the second nucleic acid molecule to produce a second strand of the double stranded nucleic acid.

12. The method of claim 11, wherein the first strand includes:
a first primer sequence;
a first identifier sequence t that includes a nucleotide sequence that is complementary to an additional nucleotide sequence that includes an identifier of the location of the individual site within the array of sites;
the first hybridization sequence;
a second identifier sequence that includes a nucleotide sequence that includes an identifier of a test compound of the one or more test compounds; and
a second primer segment.

13. The method of claim 12, wherein the second strand includes:
an additional first primer sequence that includes a nucleotide sequence that is complementary to the first primer sequence;
a third identifier sequence that includes the additional nucleotide sequence that includes the identifier of the location of the individual site;
the second hybridization sequence;
a fourth identifier sequence that includes a further nucleotide sequence that is complementary to the nucleotide sequence that includes the identifier of the test compound; and
an additional second primer sequence that includes a nucleotide sequence that is complementary to the second primer sequence.

14. The method of claim 11, comprising:
applying one or more denaturation processes to cause the first strand to separate from the second strand;
collecting the second strand; and
performing one or more sequencing operations with respect to the second strand to produce sequencing data corresponding to the second strand.

15. The method of claim 14, comprising:
analyzing sequencing reads included in the sequencing data to determine that a test compound of the one or more test compounds was present in the individual site; and
collecting electrical response data for the individual site that indicates the electrical signals produced in response to the at least one of voltage or current being applied to the one or more electrodes.

16. The method of claim 15, comprising:
analyzing the electrical response data to determine an efficacy of the test compound with respect to one or more biological conditions related to the number of biological cells.

17. The method of claim 1, comprising:
performing an enzymatic nucleic acid synthesis process or a light-based nucleic acid synthesis process to produce the one or more first nucleic acid molecules within the individual site;
wherein the one or more first nucleic acid molecules are synthesized using one or more initiator molecules that are bound to a base surface of the individual sites.

18. The method of claim 1, comprising:
after dispensing the solution on the semiconductor device that includes the plurality of compound carriers with the one or more second nucleic acid molecules coupled to the individual compound carrier and the one or more test compounds being coupled to the individual compound carrier, dispensing an oil solution on the semiconductor device;
wherein the oil solution is dispensed on the semiconductor device before applying the at least one of the voltage or the current to the one or more electrodes included in the individual site.

19. The method of claim 1, wherein the compound carrier includes a bead and a diameter of at least a portion of the individual site is no greater than 1.5 times a diameter of the bead.

20. The method of claim 1, wherein the CMOS layer includes circuitry to (i) apply at least one of the voltage or the current to the one or more electrodes included in the individual sites and (ii) measure the electrical signals produced in response to the at least one of voltage or current being applied to the one or more electrodes.
